(19) United States
(12) Patent Application Publication
(10) Pub. No.: US 2024/0042024 A1
(43) Pub. Date: Feb. 8, 2024

(54) METHODS AND COMPOSITIONS FOR TREATMENT OF IMMUNE-MEDIATED DISEASES

(71) Applicants: David A. HORWITZ, Santa Monica, CA (US); GENERAL NANOTHERAPEUTICS LLC, Santa Monica, CA (US)

(72) Inventor: David A. HORWITZ, Santa Monica, CA (US)

(21) Appl. No.: 18/254,690

(22) PCT Filed: Nov. 23, 2021
(86) PCT No.: PCT/US2021/060612

§ 371 Date: May 26, 2023

Related U.S. Application Data
(60) Provisional application No. 63/118,863, filed on Nov. 27, 2020.

(51) Int. Cl. A61K 39/00; A61K 38/20; A61K 38/17; A61K 38/18; A61P 37/06

(52) U.S. Cl. CPC A61K 39/4621; A61K 39/46434; A61K 38/2013; A61K 38/1729; A61K 38/1841; A61P 37/06; A61K 2039/5154; A61K 2039/577

(57) ABSTRACT

A novel nanoparticle platform has been developed that induces and expands multiple populations of suppressive regulatory cells in vivo for the prevention and treatment of immune-mediated disorders. These include autoimmune diseases, graft-versus-host disease, and transplant rejection. The regulatory cells expanded include both CD4+ and CD8+ T cells and NK cells. The nanoparticles function as artificial antigen-presenting cells (aAPC) that target T cells and NK cells and provide them the essential stimulation and cytokines they require for regulatory cell generation, function, and expansion. This is achieved without the use of the toxic immunosuppressive and biological agents now in use.

### CROSS REFERENCE TO RELATED APPLICATIONS

This application is a U.S. National Stage filing of International Patent Application Number PCT/US2021/060612, filed Nov. 23, 2021, which claims priority to U.S. Application No. 63/118,863, filed on Nov. 27, 2020, each of which is incorporated by reference herein in its entirety.

### FIELD OF THE INVENTION

Described herein are methods of making and using nanoparticle (NP) formulations to treat and compositions, especially nanoparticle formulations, for the treatment of immune-mediated disorders that include antibody and cell-mediated autoimmune diseases, graft versus host disease, and solid organ graft rejection.

### BACKGROUND OF THE INVENTION

Immune-mediated diseases occur when foreign or self-immune cells attack the body's own cells. They include autoimmune diseases, graft-versus-host disease and rejection of foreign solid organ transplants. Autoimmune disease happens when normally quiescent self-reactive immune cells become activated and attack the body's own cells. There are more than 80 types of autoimmune diseases that affect a wide range of body parts. Common autoimmune diseases include rheumatoid arthritis, psoriasis, psoriatic arthritis, systemic lupus erythematosus (SLE), type 1 diabetes, inflammatory bowel disease, and thyroid diseases. Graft versus host disease occurs when foreign hematopoietic stem cells are used for the treatment of hematologic malignancies. Foreign solid organ transplants are rejected without adequate immunosuppression. With unusual autoimmune diseases, patients may suffer years before getting a proper diagnosis. Most of these diseases have no cure. Some require lifelong treatment to ease symptoms. Collectively, these diseases affect more than 24 million people in the United States. An additional eight million people have autoantibodies, indicating a person's chance of developing autoimmune disease. Studies indicate these diseases likely result from interactions between genetic and environmental factors. Gender, race, and ethnicity characteristics are linked to a likelihood of developing an autoimmune disease. Autoimmune diseases are more common when people are in contact with certain environmental exposures.

Many autoimmune diseases have similar symptoms. This makes it hard for a health care provider to diagnose autoimmune disease, and then to identify the specific autoimmune disease. Often, the first symptoms are fatigue, muscle aches and a low fever. The classic sign of an autoimmune disease is inflammation, which can cause redness, heat, pain and swelling. The diseases may also have flare-ups, when they get worse, and remissions, when symptoms get better or disappear. Treatment depends on the disease, but in most cases one important goal is to reduce inflammation. Sometimes doctors prescribe corticosteroids or other drugs that reduce the immune response.

There is an urgent need to develop compositions and methods for the treatment of autoimmune diseases such as SLE. Therefore, it is the object of the disclosure herein to provide compositions for the treatment of chronic autoimmune diseases and to provide methods for the treatment of lupus, graft versus host disease, and other chronic immune-mediated diseases.

### BRIEF SUMMARY OF THE INVENTION

In one aspect, the present disclosure provides for a method of treating an immune-mediated disorder in a patient comprising administering to the patient a tolerogenic artificial Antigen Presenting Cell (aAPC) composition comprising: (i) at least one synthetic polymeric nanoparticle, (ii) at least one targeting agent, and (iii) at least one stimulating agent.

In some embodiments, the at least one targeting agent targets T cells.

In some embodiments, the at least one targeting agent targets NK cells.

In some embodiments, the at least one targeting agent targets T cells and NK cells.

In some embodiments, the at least one targeting agent targets NKT cells.

In some embodiments, the at least one targeting agent targets CD3.

In some embodiments, the at least one targeting agent targets CD2.

In some embodiments, the at least one targeting agent targets CD3 and CD2.

In some embodiments, the at least one targeting agent induces cells in the patient to produce TGF-β in the local environment.

In some embodiments, the at least one targeting agent is an antibody.

In some embodiments, the at least one targeting agent is at least one member selected from the group consisting of: an anti-CD2 antibody, an anti-CD3 antibody, and an anti-CD3 antibody with an inactivated or absent Fc fragment.

In some embodiments, the at least one targeting agent is an aptamer.

In some embodiments, the aptamer binds TCR-CD3.

In some embodiments, the at least one stimulating agent comprises a cytokine.

In some embodiments, the at least one stimulating agent comprises IL-2.

In some embodiments, the at least one stimulating agent is encapsulated.

In some embodiments, the method induces lymphocytes in the patient to become multiple populations of functional regulatory cells.

In some embodiments, the method induces both CD4 and CD8 cells in the patient to become Foxp3+ T regulatory cells.

In some embodiments, the method generates and expands regulatory NK cells to numbers that suppress the immune-mediated disorder.

In some embodiments, the method generates and expands one or more lymphocyte populations to numbers that suppress the immune-mediated disorder.

In some embodiments, NK cells in the patient become TGF-β producing regulatory NK cells

In some embodiments, T cells become TGF-β producing regulatory T cells.

In some embodiments, the cytokine is TGF-β and the TGF-β is either encapsulated in the nanoparticle or the nanoparticle induces regulatory cells in vivo in the local environment.

In some embodiments, the immune-mediated disorder is at least one antibody-mediated autoimmune disease selected from a group consisting of: systemic lupus erythematosus, pemphigus vulgaris, myasthenia gravis, hemolytic anemia, thrombocytopenia purpura, Graves disease, dermatomyositis and Sjogren's disease.

In some embodiments, the immune-mediated disorder is at least one cell-mediated autoimmune disease selected from a group consisting of type 1 Diabetes, Hashimoto's Disease, polymyositis, inflammatory bowel disease, multiple sclerosis, rheumatoid arthritis and scleroderma.

In some embodiments, the immune-mediated disorder is a graft-related disease.

In some embodiments, the immune-mediated disorder is rejection of a foreign organ transplant.

In some embodiments, the immune-mediated disorder is graft versus host disease.

In some embodiments, the method is performed in vitro.

In some embodiments, the method is performed in vivo.

In some embodiments, the administering to the patient is using parenteral delivery.

In some embodiments, the parenteral delivery is intravenous.

In some embodiments, the parenteral delivery is intramuscular.

In some embodiments, the parenteral delivery is subcutaneous.

In some embodiments, the administering to the patient is using oral delivery.

In some embodiments, the at least one synthetic polymeric nanoparticle is selected from the group consisting of: a glycide, a liposome, and a dendrimer.

In some embodiments, the aAPC is combined with at least one defensin.

In another aspect, the present disclosure provides for a method of preventing an immune-mediated disorder in a patient comprising administering to the patient a tolerogenic artificial Antigen Presenting Cell (aAPC) composition comprising: (i) at least one synthetic polymeric nanoparticle, (ii) at least one targeting agent, and (iii) at least one stimulating agent.

In some embodiments, the at least one targeting agent targets T cells.

In some embodiments, the at least one targeting agent targets NK cells.

In some embodiments, the at least one targeting agent targets T cells and NK cells.

In some embodiments, the at least one targeting agent targets NKT cells.

In some embodiments, the at least one targeting agent targets CD3.

In some embodiments, the at least one targeting agent targets CD2.

In some embodiments, the at least one targeting agent targets CD3 and CD2.

In some embodiments, the at least one targeting agent induces cells in the patient to produce TGF-β in the local environment.

In some embodiments, the at least one targeting agent is an antibody.

In some embodiments, the at least one targeting agent is at least one member selected from the group consisting of: an anti-CD2 antibody, an anti-CD3 antibody, and an anti-CD3 antibody with an inactivated or absent Fc fragment.

In some embodiments, the at least one targeting agent is an aptamer.

In some embodiments, the aptamer binds TCR-CD3.

In some embodiments, the at least one stimulating agent comprises a cytokine.

In some embodiments, the at least one stimulating agent comprises IL-2.

In some embodiments, the at least one stimulating agent is encapsulated.

In some embodiments, the method induces lymphocytes in the patient to become multiple populations of functional regulatory cells.

In some embodiments, the method induces both CD4 and CD8 cells in the patient to become Foxp3+ T regulatory cells.

In some embodiments, the method generates and expands regulatory NK cells to numbers that suppress the immune-mediated disorder.

In some embodiments, the method generates and expands one or more lymphocyte populations to numbers that suppress the immune-mediated disorder.

In some embodiments, NK cells in the patient become TGF-β producing regulatory NK cells

In some embodiments, T cells become TGF-β producing regulatory T cells.

In some embodiments, the cytokine is TGF-β and the TGF-β is either encapsulated in the nanoparticle or the nanoparticle induces regulatory cells in vivo in the local environment.

In some embodiments, the immune-mediated disorder is at least one antibody-mediated autoimmune disease selected from a group consisting of: systemic lupus erythematosus, pemphigus vulgaris, myasthenia gravis, hemolytic anemia, thrombocytopenia purpura, Graves disease, dermatomyositis and Sjogren's disease.

In some embodiments, the immune-mediated disorder is at least one cell-mediated autoimmune disease selected from a group consisting of type 1 Diabetes, Hashimoto's Disease, polymyositis, inflammatory bowel disease, multiple sclerosis, rheumatoid arthritis and scleroderma.

In some embodiments, the immune-mediated disorder is a graft-related disease.

In some embodiments, the immune-mediated disorder is rejection of a foreign organ transplant.

In some embodiments, the immune-mediated disorder is graft versus host disease.

In some embodiments, the method is performed in vitro.

In some embodiments, the method is performed in vivo.

In some embodiments, the administering to the patient is using parenteral delivery.

In some embodiments, the parenteral delivery is intravenous.

In some embodiments, the parenteral delivery is intramuscular.

In some embodiments, the parenteral delivery is subcutaneous.

In some embodiments, the administering to the patient is using oral delivery.

In some embodiments, the at least one synthetic polymeric nanoparticle is selected from the group consisting of: a glycide, a liposome, and a dendrimer.

In some embodiments, the aAPC is combined with at least one defensin.

### BRIEF DESCRIPTION OF THE DRAWINGS

FIG. 1A-1G shows that nanoparticles (NPs) coated with anti-CD2 and anti-CD4 containing IL-2 and TGF-β functioned as tolerogenic artificial antigen-presenting cells (aAPCs). They increased CD4+ and CD8+ Foxp3+ Tregs and prevented a lupus-like syndrome in (CD56/BL6×DBA2) F1 hybrid mice following the injection of parental DBA2 T cells. These NP aAPCs suppressed anti-DNA production and prevented severe renal disease. However, depletion of NK cells blocked the increase in CD4+ and CD8+ Tregs and increased the severity of lupus nephritis. FIG. 1A shows the treatment schedule and dose of tolerogenic anti-CD2/4 coated NPs (tolerogenic NP aAPCs) containing IL-2 and TGF-β given to (C56/BL6×DBA2)F1 mice following administration of DBA/2 c cells. FIG. 1B shows the increase In Vivo in CD4+CD25+Foxp3+ Tregs. FIG. 1C shows the increase in CD8+Foxp3+ Tregs. FIG. 1D shows the development of nephritis at 4 weeks indicated by proteinuria. The tolerogenic aAPCs markedly inhibited the proteinuria. However, following NK cell depletion with anti-asialo GM1 antibodies, the increase in CD4 and CD8 Tregs was inhibited and the amount of proteinuria increased significantly greater than in animals that did not receive NPs. Symbols represent the different groups of mice (n=6 per group); error bars show the mean±SEM. The percentages of peripheral CD4+(FIG. 1B) and CD8+(FIG. 10) Tregs are also shown at the indicated time points after treatment. *P<0.05 and **P<0.05 in the comparison between empty NPs versus cytokine-loaded NPs; § P<0.04 between mice depleted (anti-asialo GM1, a-asGM1) or not of NK cells. FIG. 1D shows proteinuria at the time points indicated for the mice in FIG. 2B-2C. *P<0.05 between empty NPs vs. NP aAPCs; § P<0.05 and **P<0.005 in the comparison between mice treated with cytokine-loaded NPs depleted (anti-aGM1) or not of NK cells. NK cell depletion markedly reduced the number of circulating CD4+ and CD8+ Tregs and exacerbated renal disease in nanoparticle treated mice (FIGS. 1B-2C). The panels in FIGS. 1A-1G (more specifically FIGS. 1E-1G) show that depletion of NK cells associates with increased levels of serum anti-dsDNA autoantibodies. Conversely, treatment with CD2 (NK)-targeted NPs associates with suppression of anti-DNA autoantibody production. NK cells were depleted by administering anti-asialo GM1. Monitoring of individual mice and group means are reported at week 2 and 4 post-induction of SLE (time 0). *P<0.05, **P<0.01.

FIGS. 2A-2D: FIGS. 2A-2B show that host NK cells expand numerically in BDF1 mice with lupus-like disease after treatment with CD2-targeted NPs loaded with IL-2 and TGF-β. Controls were uncoated NPs loaded with IL-2 and TGF-β and empty uncoated NPs. The top two panels (FIG. 2A and FIG. 2B) are total amounts of NPs per treatment, the bottom two (FIG. 2C and FIG. 2D) are the doses of NPs each time.

FIGS. 2A-2D: FIGS. 2A-B show increases in NK cells during the four weeks after administration of the NP aAPCs. FIG. 2A shows the increased percentages and FIG. 2B the absolute increase in NK cells at each week. The symbols show the total dose of NP aAPCs given. FIG. 2B shows the total numbers of NK cells with mean+SE in mice with the same treatments. P values in the comparison with SLE BDF1 mice treated with empty NPs=*<0.005, **0.0005. FIGS. 2C-2D. show the dose of NPs given at each injection in individual mice. The symbols show untreated BDF1 mice (“Non-SLE”, squares) or lupus BDF1 mice treated with different doses of NPs encapsulating IL-2 and TGF-β (circle, uncoated NPs; triangle 1 mg; diamond 2 mg; inverted triangle 4 mg). These increases are also statistically significant.

FIG. 3 shows that following administration of NP aAPCs there was a marked increased in host-derived NK cells. Monitoring by flow cytometry after treatment with NPs loaded with IL-2 and either left uncoated (control) or coated with anti-CD2 antibodies. The use of H-2 markers allowed discrimination of the NK cells (NK1.1+) from DBA/2 donors (H-2Kb−) vs. BDF1 recipients (H-2Kb+).

FIGS. 4A-4C show protection from lupus nephritis of BDF1 mice treated with CD2 (NK)-targeted NPs depends on NK cells and TGF-β. A novel TGF-β-dependent regulatory NK cell is described. FIG. 4A shows that NK cell depletion accelerates proteinuria in BDF1 mice. NK cells were depleted by administering 100 ul anti-asialo GM1 every 4 days for 2 weeks from day 0 (induction of SLE). Mice (n=6 per group) were monitored for 8 weeks post-induction of SLE. Data show the Data show the mean+SE; *P<0.01 at 4 and 6 weeks in the comparison between BDF1 mice receiving NK cell-targeted NPs with or without NK-depleting anti-asialo GM1 and at 4 weeks between mice receiving empty, non-targeted NPs vs. mice depleted of NK cells. FIG. 4B shows that administration of anti-TGF-β antibodies, anti-asialo GM1 antibodies or a combination of both to BDF1 lupus mice (n=6 per group) abrogates the NK cell-mediated protective effects associated with treatment with anti-CD2-targeted NPs. Renal function was assessed by the serum creatinine. This was measured 2 weeks after treatment with anti-TGF-β or control (ctr) Ab. *P<0.04. FIG. 4C shows that the protective effect of the NK cells is TGF-β dependent. Following treatment of mice with the anti-CD2 targeted aAPCs, NK cells were isolated. Some were treated with TGF-β siRNA and others with RNA scrambled control. 2.5×10⁶ NK cells of each set were transferred into syngeneic BDF1 mice which were then induced to develop lupud. Serum creatinine was measured 2 weeks after transfer. *P<0.01. NK cells from mice that had received aAPCs prevented the development of chronic renal disease in secondary hosts. The increase in serum creatine in mice where NK cells TGF-β message was silenced indicates that protective effect of these NK cells was TGF-β dependent.

FIGS. 5A-5B show that the aAPCs could also expand human Tregs. PBMCs from healthy volunteers cultured for 5 days with anti-CD3/28 beads at a ratio of 0.2 beads/cell. Experimental cultures included 100 μg/ml NPs loaded with IL-2 and TGF-β, either left uncoated or decorated with antibodies to T cells (anti-CD3/28). Cultures with medium only and either no NPs (unstimulated) or NPs kept unloaded (empty) served as negative controls; cultures with anti-CD3/28 in the presence of soluble IL-2 and TGF-β served as positive control. The addition of these NPs functioned as tolerogenic aAPCs and increased CD4+CD25hiCD127-FoxP3+ Tregs (FIG. 5A) and CD8+FoxP3+ Tregs (FIG. 5B) P<0.05.

FIGS. 6A-6B show that like mouse cells, NPs containing IL-2 only can induce CD4 and CD8 Tregs. Human PBMCs were cultured with NPs targeted to T cells decorated with anti-CD3 and CD28 for the induction of Tregs. FIG. 6A shows T-cell-targeted NPs that only encapsulated IL-2 promoted the expansion of CD4+ and CD8+ Tregs. *P<0.05. FIG. 6B shows CD4+ Tregs induced by these aAPC NPs targeted to T cells suppressed in vitro the proliferation (left) and IFN-γ production (right) of cocultured CD4+CD25− T cells. *P<0.05 in the comparison with Treg:Teff at the 0:1 ratio (only stimulated T effector cells).

FIGS. 7A-7C are graphs showing that T cell-targeted tolerogenic aAPC NPs can also expand human Tregs in vivo. Immunodeficient NOD/SCID mice (NSG) were humanized by transfer of human PBMC. The administration of NP tolerogenic aAPC expanded human Tregs and suppressed the GVHD. NSG mice were divided into two groups of 6 mice each. Following transfer of PBMCs, solid circles indicate mice given aAPC NPs and open circles were mice given empty NPs. FIGS. 7A and 7B show marked increase in both CD4 and CD8 Tregs following transfer of the aAPC. Although the NPs were given during the first two weeks, remarkably, increased levels of both CD4 and CD8 Tregs remained detectable when the experiment was concluded on day 50 (*=p<0.5). FIG. 7C shows evidence of human B cell activity in these mice. There was a marked rise in human IgG levels in mice that received empty NPs that was not observed in the mice that received the aAPCs (*=p<0.5).

FIGS. 8A-8E: FIGS. 8A-8C are graphs showing the protective effects of Tregs induced and expanded by the administration of T cell-targeted tolerogenic aAPC NPs to immunodeficient NOD/SCID mice (NSG) following humanization by transfer of human PBMC. FIGS. 8A-8E show the effects of the aAPC on the human anti-mouse GVHD. X marked lines show control mice that did not receive human PBMC. Triangles show mice that received empty NPS, and open circles mice that received aAPC NPs. The aAPC NP-protected mice did not lose weight after transfer of the human PBMCs (FIG. 8A), the disease score was decreased (FIG. 8B), and the treated mice had an extended survival (FIG. 8C) as compared to the mice that had not received NPs or that had received empty NPs. * show statistically significant results between the two groups (p<0.05). The control mice developed the cutaneous manifestations of GVHD compared with controls (FIG. 8D). FIG. 8E shows the inflammatory infiltrate in lung, liver and colon compared to controls.

FIGS. 9A and 9B show that anti-CD2 and anti-CD3 coated NPs do not need to contain TGF-β to induce human Tregs. They provide the TGF-β the local environment. Human PBMC (0.5×105)/well were cultured in U-bottom plates for 5 days. Biotinylated anti-CD2, anti-CD3), or a combination of both (2 ul/ml) was attached to the surface of PLGA NPs. 50 ug/ml of these NPs containing both IL-2 and TGF-β or IL-2 alone were added to the PBMC. Without other stimulation, NPs with and without TGF-β induced at least a 2-fold increase in CD4regs and a 4-fold increase in CD8regs (FIGS. 9A and B). The addition of anti-TGF-β to the cultures abolished this increase. This result indicated that both IL-2 and TGF-β was needed to induce the Tregs and that the NPs induced exogenous TGF-β needed to induce the Tregs. It is known that anti-CD3 can induce T cells to produce this cytokine and anti-CD2 can also induce NK cells to TGF-β. Alternatively, the acidic NPs in the local environment can convert latent TGF-β present to its active form. These studies, then, provide evidence that TGF-β does not need to be encapsulated in the NPs.

FIGS. 10A and 10B show that anti-CD3 (Fab′)2 coated NPs containing IL-2 only can induce human CD4 and CD8 Tregs. Since Tregs in the periphery are induced primarily from naïve T cells, PBMC were depleted of CD45RO+ cells with magnetic beads (through AutoMACS). These cells were cultured with 200 μg/ml NPs encapsulating IL-2 and coated with anti-CD3 F(ab′)2 (x axis) or nothing (control, none) at a concentration of 5×105 cells/well in U-bottom 96-well plates in complete medium. The graphs show increased numbers of FoxP3+ cells within the CD4+ and CD8+ T cell compartments after 5 days of culture. *P<0.01.

FIGS. 11A and 11B show that although lymphocyte targeted NPs containing IL-2 only can protect immunodeficient mice from human anti-mouse graft versus host disease, the data also shows that protection is dependent on the production of TGF-β. Panel A shows the previous protective effect of administration of NPs loaded with IL-2 and TGF-β on human anti-mouse GVHD. Survival curves with x-labeled lines indicate controls that received empty NPs. Lines with solid circles indicate increased survival by animals that received IL-2 and TGF-β NPs. Lines with asterisks show that blocking TGF-β signaling with alk5 inhibitors not only abolish the protective effects of the NPs, but also shorten survival. Panel B shows that NPs containing only IL-2 have similar protective effects that are blocked by inhibiting TGF-β signaling.

FIG. 12 shows antiDNA IgG (O.D.) at week 2 and week 4 for BDF1 mice with lupus-like disease treated with anti-CD2 (NK)-targeted NPs loaded with IL-2. The methods used in this experiment are identical to the methods described in FIG. 1.

### DETAILED DESCRIPTION OF THE INVENTION

### I. Introduction

Systemic lupus erythematosus (“SLE”) is a disorder of immune regulation where genetic and environmental factors contribute to the disruption of immune homeostasis. In SLE, normally quiescent self-reactive T and B cells become activated and are no longer held in check by mechanisms of peripheral tolerance, including the suppression by T regulatory (Treg) cells, which are specialized cells that have an impaired function in SLE (Ferretti and La Cava, Overview of the pathogenesis of systemic lupus erythematosus, In: Tsokos (Ed.), Systemic lupus erythematosus. Basic, applied and clinical aspects. Cambridge: Academic Press; 2016, 55-62).

Presently, the treatment of SLE (and other autoimmune diseases) includes agents that target proinflammatory cytokines, effector cells, or signaling pathways (Wong et al., Drugs Today (Barc), 2011; 47:289-302). Although those agents can block disease progression, they rarely induce remission because they also target the compensatory regulatory pathways that are required to stop disease. Other attempts to treat SLE have tried to “reset” the immune system to cause remission. For example, lymphoid cell depletion followed by autologous stem cell transplantation results in extended disease remission in SLE but this strategy is associated with postoperative patient mortality (Burt et al., JAMA, 2006; 295:527-35).

Multiple approaches that use ex vivo-expanded CD4+ Treg cells in autoimmune diseases are under investigation, especially in type 1 diabetes (Gitelman et al., J Autoimmun, 2016; 71:78-87), but have disadvantages such as the need for autologous Treg cells that remain functionally stable in vivo, in addition to requiring technically cumbersome procedures to prepare Treg cells in large numbers. The adoptive transfer of regulatory CD8 cells and NK cells, as well as tolerogenic antigen-presenting cells, could have therapeutic effects, but the methodology for the utilization of these cells has not been developed.

Nanoparticles (NPs) targeted to T cells or antigen-presenting cells (APCs) have been used to induce immune tolerance. Polymeric NPs encapsulating tolerogenic peptides or peptides and rapamycin that targeted APCs, prevented/reversed disease in animal models of autoimmune diabetes and multiple sclerosis through TGF-β-dependent induction of Tregs (Hunter et al., ACS Nano, 2014; 25:2148-60; Maldonado et al., Proc Natl Acad Sci USA, 2015; 112:156-65). NP-mediated delivery of immunosuppressive drugs of Ca2+/calmodulin-dependent protein kinase IV (CaMK4) inhibitor ameliorated murine SLE (Look et al., J Clin Invest, 2013, 123:1741-9; Otomo et al., J Immunol, 2015; 195:5533-7; Maeda et al., J Clin Invest, 2018; 128:3445-59).

Iron oxide NPs coated with MHC-peptides can convert IFN-γ-producing Th1 cells into IL-10-producing Tr1 cells, affording therapeutic effects in mouse models of autoimmune disease (Clemente-Casares et al., Nature, 2016; 530:434-40). Although CD4+ Tregs induced dendritic cells (DCs) to become tolerogenic and protect secondary hosts (Lan et al., J Mol Cell Biol, 2012; 4:409-19), that system had limitations, including not allowing delivery of multiple therapeutic agents, and different MHC-specific peptides would be needed to match the MHC diversity encountered in human autoimmune diseases. Also, extended iron oxide accumulation is toxic.

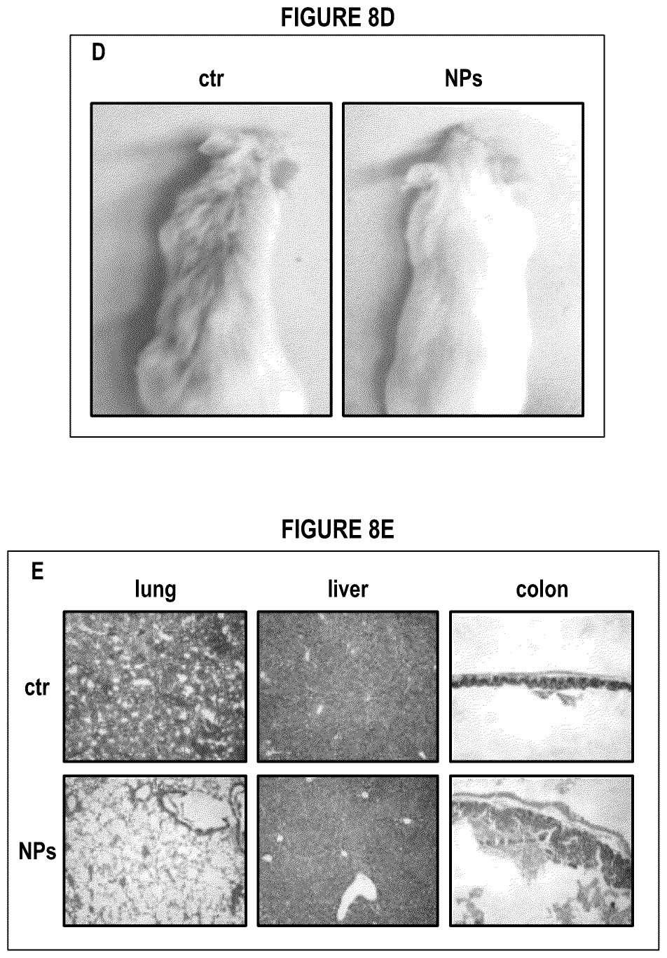

The present application provides methods and compositions to directly induce multiple populations of immune cells to become cells that prevent, or treat established immune-mediated disorders have been developed. Since T cells cannot respond to antigen directly, nanoparticles acting as tolerogenic artificial antigen-presenting cells (aAPC) that can fully induce an immature cell to become a suppressive, regulatory cells are administered. For T cells and NK cells, this requires continuous stimulation, using IL-2 and TGF-β. The aAPC provides all three elements: T cell receptor (TCR) stimulation, IL-2 and TGF-β. It has now been discovered that encapsulating IL-2 alone can be used (FIGS. 9-11). Anti-CD2 and CD3 can induce the TGF-β needed for T cells in the local environment. Anti-CD2 can also induce NK cells to produce TGF-β and induces them to become TGF-β-dependent regulatory cells.

Both anti-CD3, and anti-CD2 coated NPs loaded with IL-2 can provide the stimulation and cytokines needed for generation and proliferation of the regulatory cells, but the mechanisms may be different. A method to generate these cells in vivo with NP aAPCs provides a safe, practical therapeutic approach for multiple indications of immune-mediated diseases.

T cells are unable to respond directly to antigen-stimulation, and need an antigen presenting cell (APC) for this purpose. Nanoparticles can serve as tolerogenic artificial antigen-presenting cells or aAPCs. Previously Park et al (Mol Pharm, 2011; 8:143-52), coated PLGA NPs with anti-CD3 and anti-CD28 and loaded the NPs with IL-2 to create an immunogenic aAPC. Thus, NPs can be formulated to become either immunogenic or tolerogenic aAPCs.

While NPs can target APC to expand Tregs they can also directly induce or expand Tregs. T cell differentiation is determined in part by the strength of T cell receptor stimulation. While strong stimulation is immunogenic and produces T effector cells, weaker stimulation through the identical pathway can be tolerogenic and produce T regulatory cells. Thus, altering the composition of the antibodies coating the NPs can switch immunogenic aAPCs to the tolerogenic aAPCs described in this document.

The most protective Tregs are CD4+CD25+Foxp3+ cells. These Tregs require continuous stimulation and the cytokines IL-2 and TGF-β for their induction, fitness and survival (Sakaguchi S et al., Immunol Rev, 2006; 212:8-27). In SLE these Tregs are dysfunctional. Production of IL-2 and TGF-β is also decreased in lupus and it is likely that this defect contributes to Treg dysfunction. Therefore, a method that provides immune cells IL-2 and TGF-β in vivo could correct the IL-2 defect in lupus and induce and expand therapeutic Tregs in lupus. However, TGF-β has pleotropic properties that could cause adverse side effects. It is therefore desirable to use nanoparticles that induce exogenous TGF-β in the local environment for this effect.

A tolerogenic nanoparticle platform has been developed that expands both CD4+ and CD8+ T regulatory (Treg) cells and induces a TGF-β dependent natural killer (NK) regulatory response in vivo. These multiple regulatory cell populations suppress chronic immune-mediated diseases that include autoimmune disorders such as systemic lupus erythematosus (SLE) and include foreign transplantation disorders such as graft-versus-host disease.

As indicated herein, T cells cannot respond to antigen directly. They require antigen-presenting cells (APCs) to induce them to differentiate into positive effector cells or negative suppressor or regulatory cells. The regulatory cells modulate effector cell activity and prevent quiescent self-reactive cells from causing autoimmunity. Nanoparticles have been formulated to become tolerogenic artificial antigen-presenting cells (aAPC) that target T cells and natural killer (NK cell). Methods have been developed to use these that aAPCs to induce and expand CD4+ and CD8+ and NK regulatory cells in vitro and in vivo. The platform provides the cytokines IL-2, TGF-β and the continuous stimulation that is essential for the generation, function and survival of these regulatory cell population.

The nanoparticles encapsulate IL-2 for release, and are preferably targeted to cells expressing CD2 and/or CD3 using antibodies that coat the NPs. Anti-CD2 targets T cells and NK cells while anti-CD3 targets only T cells. The immune stimulation provided by these antibodies and the effects of IL-2 released by the NPs induce the T cells and NK cells to produce TGF-β, or activate latent TGF-β present in the local environment. The cumulative effects of the stimulation and the cytokines produced induce undifferentiated CD4+ and CD8+ T cells to become Tregs and NK cells to become TGF-β-dependent regulatory cells which have therapeutic effects on immune-mediated diseases.

Anti-CD3 injected in vivo can result in toxic side effects which include cytokine release syndrome. These side effects are mediated by the Fc portion of the antibody. To eliminate this toxicity, the Fc fragment of this antibody can be eliminated without altering therapeutic properties.

Several examples show that or anti-CD3 (Fab′)2 or anti-CD2 coated NPs loaded with only IL-2 possess the ability to induce regulatory cells. To illustrate that the NPs have induced the targeted cells to produce TGF-β, one example shows that antibody neutralization of TGF-β abolishes their ability to induce CD4+ and CD8+ Tregs. Another example shows that anti-CD2 coated NPs containing only IL-2 induce a therapeutic TGF-β-dependent NK cells. Because of the many pleotropic effects of TGF-β, this modification to produce this cytokine locally should markedly improve the safety of these NPs when used as therapeutic. It will not be necessary to encapsulate TGF-β in the nanoparticles.

Efficacy of the system was first demonstrated using a systemic lupus erythematosus (“SLE”) animal model. Poly(lactic-co-glycolic) acid (PLGA) nanoparticles (NPs) encapsulating IL-2 and TGF-β were initially coated with anti-CD2/CD4 antibodies and administered to mice with lupus-like disease induced by the transfer of DBA/2 T cells into (C57BL/6×DBA/2)F1 (BDF1) mice. DBA/2 T cells stimulate parental B cells to produce antibodies that cause a lethal lupus-like disease. Following NP administration peripheral frequency of Tregs was monitored ex vivo by flow cytometry. Disease progression was assessed by measuring serum anti-dsDNA antibodies by ELISA. Nephritis was evaluated as proteinuria and renal histopathology.

Anti-CD2/4 antibody-coated, but not non-coated, NPs encapsulating IL-2 and TGF-β induced CD4+ and CD8+Foxp3+ Tregs in vitro. In vivo studies in normal mice determined the dosing regimen of NPs for the expansion of CD4+ and CD8+ Tregs tested in BDF1 mice with lupus. The administration of anti-CD2/CD4 antibody-coated NPs encapsulating IL-2 and TGFβ resulted in the expansion of CD4+ and CD8+ Tregs, a marked suppression of anti-DNA antibody production, and reduced renal disease.

Not only CD4+ and CD8+ Tregs were involved in the treatment. TGF-β-dependent NK regulatory cells were also involved. Mice that had been treated with anti-CD2/4 bound NPs were treated with anti-asialoGM1 antibodies to deplete NK cells. This treatment not only decreased the number of CD4+ and CD8+ Tregs induced by the NPs and completely abolished their therapeutic effects, but also increased the severity of autoimmune disease. Titers of anti-DNA antibodies were higher than in untreated mice and the renal disease (proteinuria) was greater than in untreated mice. Thus, in addition to the increased CD4+ and CD8+Foxp3+ Tregs induced by the NPs, protective NK cells were also apparently induced, and depletion of these cells completely overcame the protective effects of the NPs and exacerbated the manifestations of lupus.

In addition to studies with mouse cells, the tolerogenic aAPCs containing IL-2 and TGF-β or IL-2 alone induced human T cells to become Tregs in vitro and in vivo. Examples were PLGA NPs coated with anti-CD2, anti-CD3, and anti-CD3+anti-CD28 (which provided additional co-stimulation). NPs coated with these antibodies induced CD4+ and CD8+ Foxp3+ Tregs In Vitro. Moreover, when immunodeficient mice were transfused with human PBMC and given aAPCs encapsulating IL-2 only for three weeks, there was a marked increase in CD4+ and CD8+ Foxp3+ Tregs in vivo that persisted for 5 months and the protective effects of these regulatory cells enabled most of these mice to survive the ensuing lethal human anti-mouse graft disease.

These results highlight the use of this technology in human systemic autoimmune disease. In autoimmune diseases the TCR stimulation is from the autoantigen. In autoimmune diseases such as SLE, type 1 diabetes and multiple sclerosis, pathogenic peptides have been described which can be converted into tolerogenic peptides when incorporated into the aAPC NPs. In allogeneic stem cell transplantation and allotransplants the foreign alloantigens are processed by immunogenic antigen-presenting cells and presented to T cells which become killer cells that cause graft-versus host disease or transplant rejection. For this reason, toxic immunosuppressive drugs are employed before the graft to eliminate the immune cells that mediate rejection. It would be desirable to eliminate this toxic conditioning procedure and the immunosuppressive drugs needed to prevent rejection following the transplant. The direct effects of tolerogenic aAPCs on lymphocytes to induce Tregs can achieve these objectives.

To avoid rejection of allogeneic organ grafts, treatment with these aAPC nanoparticles before the transplant will generate CD4 Tregs, CD8 Tregs and TGF-β-dependent NK regulatory cells. These will interact with immature antigen-presenting dendritic cells and induce them to become tolerogenic. Thus, post-transplant the aAPCs will support the tolerogenic dendritic cells that process the transplant foreign alloantigens and induce alloantigen-specific Tregs that facilitate transplant survival instead of T killer cells that reject the transplant. The methods described herein, therefore, will markedly reduce or eliminate the use of toxic corticosteroids and immunosuppressive drugs now used for allogeneic stem cell and solid organ transplants. NP aAPCs can be used for treatment or prevention of GVHD and solid organ transplantation indications.

### II. Definitions

By immune response herein is meant host responses to foreign or self-antigens. The terms “aberrant immune response” or “immune-mediated disorder” as used herein are interchangeable and mean the failure of the immune system to distinguish self from non-self or failure protect the host from foreign antigens. In other words, aberrant immune responses or immune-mediated disorders are inappropriately regulated immune responses that lead to patient symptoms. By “inappropriately regulated” is meant inappropriately induced, inappropriately suppressed and/or non-responsiveness. Aberrant immune responses include, but are not limited to tissue injury and inflammation caused by the production of antibodies to an organism's own tissue, impaired production of IL-2, IL-10 and TGF-β, excessive production of TNF-α, and IFN-γ, and tissue damage caused by cytotoxic and non-cytotoxic mechanisms of action. In all these events pathologic immune cells escape control by other immune cells that normally negatively regulate the pathologic cells to keep them silent. Accordingly, in a preferred embodiment, the present invention uses formulated nanoparticles that target specific immune cells, induce them to become suppressive regulatory cells, expands their numbers. These regulatory cells “reset” the immune system and terminate the activity of pathologic immune cells. The regulatory composition that induces T cells to become regulatory cells includes agents that provide continuous stimulation and the cytokines IL-2 and TGF-β.

“Interleukin-2” (IL-2) as described herein, is an interleukin, a type of cytokine signaling molecule in the immune system. It is a 15.5-16 kDa protein that regulates the activities of white blood cells (leukocytes, often lymphocytes) that are responsible for immunity. IL-2 is part of the body's natural response to microbial infection, and in discriminating between foreign (“non-self”) and “self”. IL-2 mediates its effects by binding to IL-2 receptors, which are expressed by lymphocytes. The major sources of IL-2 are activated CD4+ T cells and activated CD8+ T cells. IL-2 is a member of a cytokine family, each member of which has a four alpha helix bundle; the family also includes IL-4, IL-7, IL-9, IL-15 and IL-21. IL-2 has essential roles in key functions of the immune system, tolerance and immunity, primarily via its direct effects on T cells. In the thymus, where T cells mature, it prevents autoimmune diseases by promoting the differentiation of certain immature T cells into regulatory T cells, which suppress other T cells that are otherwise primed to attack normal healthy cells in the body. IL-2 also promotes the differentiation of T cells into effector T cells and into memory T cells when the initial T cell is also stimulated by an antigen, thus helping the body fight off infections. Together with other polarizing cytokines, IL-2 stimulates naive CD4+ T cell differentiation into Th1 and Th2 lymphocytes while it impedes differentiation into Th17 and follicular Th lymphocytes. Its expression and secretion is tightly regulated and functions as part of both transient positive and negative feedback loops in mounting and dampening immune responses. Through its role in the development of T cell immunologic memory, which depends upon the expansion of the number and function of antigen-selected T cell clones, it plays a key role in enduring cell-mediated immunity.

IL-2 signals through the IL-2 receptor, a complex consisting of three chains, termed alpha (CD25), beta (CD122) and gamma (CD132). The gamma chain is shared by all family members. The IL-2 receptor (IL-2R) a subunit binds IL-2 with low affinity (Kd˜10-8 M). Interaction of IL-2 and CD25 alone does not lead to signal transduction due to its short intracellular chain but has the ability (when bound to the β and γ subunit) to increase the IL-2R affinity 100-fold. Heterodimerization of the β and γ subunits of IL-2R is essential for signaling in T cells. IL-2 can signalize either via intermediate-affinity dimeric CD122/CD132 IL-2R (Kd˜10-9 M) or high-affinity trimeric CD25/CD122/CD132 IL-2R (Kd˜10-11 M). Dimeric IL-2R is expressed by memory CD8+ T cells and NK cells, whereas regulatory T cells and activated T cells express high levels of trimeric IL-2R. Various forms of IL-2, including variants of IL-2 that minimize stimulation of non-Tregs, may be used in the present disclosure.

“Transforming growth factor β” (TGF-β), a pleiotropic polypeptide, regulates multiple biological processes, including embryonic development, adult stem cell differentiation, immune regulation, wound healing, and inflammation. TGF-β family members are synthesized as prepropeptide precursors and are then processed and secreted as homodimers or heterodimers. Most ligands of this family signal through transmembrane serine/threonine kinase receptors and Smad proteins to regulate cellular functions. Alterations of specific components of the TGF-β-signaling pathway may contribute to a broad range of pathologies such as cancer, autoimmune diseases, tissue fibrosis, and cardiovascular pathology. TGF-β belongs to a family of closely related polypeptides with various degrees of structural homology and important effects on cell function. Transforming growth factor β (TGF-β) family members signal via heterotetrameric complexes of type I and type II dual specificity kinase receptors. The activation and stability of the receptors are controlled by posttranslational modifications, such as phosphorylation, ubiquitylation, sumoylation, and neddylation, as well as by interaction with other proteins at the cell surface and in the cytoplasm. Activation of TGF-β receptors induces signaling via formation of Smad complexes that are translocated to the nucleus where they act as transcription factors, as well as via non-Smad pathways, including the Erk1/2, JNK and p38 MAP kinase pathways, and the Src tyrosine kinase, phosphatidylinositol 3′-kinase, and Rho GTPases. Binding of a TGF-β family member induces assembly of a heterotetrameric complex of two type I and two type II receptors. There are seven human type I receptors and five type II receptors; individual members of the TGF-β family bind to characteristic combinations of type I and type II receptors. The receptors have rather small cysteine-rich extracellular domains, a transmembrane domain, a juxtamembrane domain, and a kinase domain; however, except for the BMP type II receptor and in contrast to tyrosine kinase receptors, the parts carboxy terminal of the kinase domains are very short. Ligand-induced oligomerization of type I and type II receptors promotes type II receptor phosphorylation of the type I receptor in a region of the juxtamembrane domain that is rich in glycine and serine residues (GS domain), causing activation of its kinase. The activated type I serine/threonine kinase receptors in turn phosphorylate members of the receptor-activated (R)-Smad family; thus, TGF-β, activin, and nodal generally induce phosphorylation of Smad2 and 3, whereas BMPs generally phosphorylate Smad1, 5, and. Activated R-Smads then form trimeric complexes with the common mediator Smad4, which are translocated to the nucleus where they cooperate with other transcription factors, coactivators, and corepressors to regulate the expression of specific genes. There are also non-Smad signaling pathways activated by TGF-β family members, including the Erk1/2, JNK, and p38 MAP kinase pathways, the tyrosine kinase Src, phosphatidylinositol-3′ (PI3)-kinase, and Rho GTPases.

As used herein the terms “biocompatible” and “biologically compatible” generally refer to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient.

As used herein the term “biodegradable polymer” generally refers to a polymer that will degrade or erode by enzymatic action and/or hydrolysis under physiologic conditions to smaller units or chemical species that are capable of being metabolized, eliminated, or excreted by the subject. The degradation time is a function of polymer composition, morphology, such as porosity, particle dimensions, and environment.

As used herein the term “amphiphilic” refers to a property where a molecule has both a hydrophilic portion and a hydrophobic portion. Often, an amphiphilic compound has a hydrophilic portion covalently attached to a hydrophobic portion. In some forms, the hydrophilic portion is soluble in water, while the hydrophobic portion is insoluble in water. In addition, the hydrophilic and hydrophobic portions may have either a formal positive charge, or a formal negative charge. However, overall they will be either hydrophilic or hydrophobic. An amphiphilic compound can be an amphiphilic polymer, such that the hydrophilic portion can be a hydrophilic polymer, and the hydrophobic portion can be a hydrophobic polymer.

As used herein, the terms “average particle size” or “mean particle size,” refer to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

As used herein, the term “pharmaceutically acceptable” refers to compounds, carriers, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the terms “encapsulated” and “incorporated” are art-recognized when used in reference to one or more agents, or other materials, incorporated into a polymeric composition. In certain embodiments, these terms include incorporating, formulating, or otherwise including such agent into a composition that allows for release, such as sustained release, of such agent in the desired application. The terms contemplate any manner by which an agent or other material is incorporated into a polymeric particle, including for example: attached to a monomer of such polymer (by covalent, ionic, or other binding interaction), physical admixture, enveloping the agent in a coating layer of polymer, and having such monomer be part of the polymerization to give a polymeric formulation, distributed throughout the polymeric matrix, appended to the surface of the polymeric matrix (by covalent or other binding interactions), encapsulated inside the polymeric matrix, etc. The term “co-incorporation” or “co-encapsulation” refers to the incorporation of more than one active agent or other material and at least one other agent or other material in a subject composition.

As used herein the terms “inhibit” and “reduce” refer to reducing or decreasing activity, expression, or a symptom. This can be a complete inhibition or reduction of in activity, expression, or a symptom, or a partial inhibition or reduction. Inhibition or reduction can be compared to a control or to a standard level.

As used herein the terms “treatment” or “treating” refer to administering a composition to a subject or a system to treat one or more symptoms of a disease. The effect of the administration of the composition to the subject can be, but is not limited to, the cessation of a particular symptom of a condition, a reduction or prevention of the symptoms of a condition, a reduction in the severity of the condition, the complete ablation of the condition, a stabilization or delay of the development or progression of a particular event or characteristic, or minimization of the chances that a particular event or characteristic will occur.

As used herein the terms “prevent”, “preventing”, “prevention”, and “prophylactic treatment” refer to the administration of an agent or composition to a clinically asymptomatic individual who is at risk of developing, susceptible, or predisposed to a particular adverse condition, disorder, or disease, and thus relates to the prevention of the occurrence of symptoms and/or their underlying cause.

As used herein the term “agent” refers to a physiologically or pharmacologically active substance that acts locally and/or systemically in the body. An active agent is a substance that is administered to a patient for the treatment (e.g., therapeutic agent), prevention (e.g., prophylactic agent), nutrition supply (e.g., nutraceutical), or diagnosis (e.g., diagnostic agent) of a disease or disorder. The term also encompasses pharmaceutically acceptable, pharmacologically active derivatives of agents, including, but not limited to, salts, esters, amides, prodrugs, active metabolites, and analogs.

As used herein the term “small molecule” generally refers to an organic molecule that is less than about 2000 g/mol in molecular weight, less than about 1500 g/mol, or less than about 1000 g/mol.

As used herein the term “immunomodulator” refers to an agent that modulates an immune response to an antigen but is not the antigen or derived from the antigen. “Modulate”, as used herein, refers to inducing, enhancing, suppressing, tolerizing, directing, or redirecting an immune response.

As used herein the terms “effective amount” and “therapeutically effective amount,” used interchangeably, as applied to the nanoparticles, therapeutic agents, and pharmaceutical compositions described herein, refer to the quantity necessary to render the desired therapeutic result. For example, an effective amount is a level effective to treat, cure, or alleviate the symptoms of a disease for which the composition and/or therapeutic agent, or pharmaceutical composition, is/are being administered. Amounts effective for the particular therapeutic goal sought will depend upon a variety of factors including the disease being treated and its severity and/or stage of development/progression; the bioavailability and activity of the specific compound and/or antineoplastic, or pharmaceutical composition, used; the route or method of administration and introduction site on the subject.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approximately +/−10%.

### III. Formulations

A. Nanoparticles

Nanoparticles used in the present disclosure have an average diameter between about 40 nm and about 500 nm, between about 60 and about 450 nm, between about 100 nm and about 400 nm, between about 100 nm and about 350 nm, or between about 100 nm and about 300 nm, such as about 150 nm, about 200 nm, about 250 nm, about 300 nm, or about 350 nm. The particle size may be measured with any suitable method. Suitable methods include dynamic light scattering (DLS), cryogenic-transmission electron microscopy (cryo-TEM), small angle x-ray scattering (SAXS), or small angle neutron scattering (SANS).

1. Synthetic Polymeric Nanoparticles

The polymeric matrix of the nanoparticle may be formed from one or more polymers, copolymers or blends and dendrimers. By varying the composition and morphology of the polymeric matrix, one can achieve a variety of controlled release characteristics, permitting the delivery of moderate constant doses of one or more active agents over prolonged periods of time. Preferably, the polymeric matrix is biodegradable. The polymeric matrix can be selected to degrade within a time period between one day and one year, more preferably between one day and 26 weeks, more preferably between one days and 20 weeks, most preferably between one day and 4 weeks. In some aspects, the polymeric matrix can be selected to degrade within a time period between few hours and 5 weeks, more preferably between one day and 3 weeks, more preferably between one day and 15 days, most preferably between one day and seven days.

In general, synthetic polymers are preferred, although natural polymers may be used. Representative polymers include polyhydroxy acids (poly(lactic acid), poly(glycolic acid), poly(lactic acid-co-glycolic acids)), polyhydroxyalkanoates such as poly3-hydroxybutyrate or poly4-hydroxybutyrate, polycaprolactones; poly(orthoesters); polyanhydrides; poly(phosphazenes); poly(lactide-co-caprolactones); poly(glycolide-co-caprolactones); polycarbonates such as tyrosine polycarbonates; polyamides (including synthetic and natural polyamides), polyvinyl alcohols, polyvinylpyrrolidone; poly(alkylene oxides) such as polyethylene glycol (PEG) and pluronics (polyethylene oxide polypropylene glycol block copolymers), polyacrylic acids, as well as derivatives, copolymers, and blends thereof.

As used herein, “derivatives” include polymers having substitutions, additions of chemical groups and other modifications to the polymeric backbones described above routinely made by those skilled in the art. Natural polymers, including proteins such as albumin, collagen, gelatin, prolamines, such as zein, and polysaccharides such as alginate and pectin, may also be incorporated into the polymeric matrix. In certain cases, when the polymeric matrix contains a natural polymer, the natural polymer is a biopolymer which degrades by hydrolysis.

In some embodiments, the polymeric matrix of the core particle may contain one or more crosslinkable polymers. The crosslinkable polymers may contain one or more photo-polymerizable groups, allowing for the crosslinking of the polymeric matrix following particle formation. Examples of suitable photo-polymerizable groups include vinyl groups, acrylate groups, methacrylate groups, and acrylamide groups. Photo-polymerizable groups, when present, may be incorporated within the backbone of the crosslinkable polymers, within one or more of the sidechains of the crosslinkable polymers, at one or more of the ends of the crosslinkable polymers, or combinations thereof.

The polymeric matrix of the core particle may be formed from polymers having a variety of molecular weights, so as to form particles having properties, including drug release rates, effective for specific applications.

In some embodiments, the polymeric matrix is formed from an aliphatic polyester or a block copolymer containing one or more aliphatic polyester segments. Preferably the polyester or polyester segments are poly(lactic acid) (PLA), poly(glycolic acid) PGA, or poly(lactide-co-glycolide) (PLGA). The degradation rate of the polyester segments, and often the corresponding drug release rate, can be varied from days (in the case of pure PGA) to months (in the case of pure PLA), and may be readily manipulated by varying the ratio of PLA to PGA in the polyester segments. In addition, PGA, PLA, and PLGA have been established as safe for use in humans; these materials have been used in human clinical applications, including drug delivery system, for more than 30 years.

Examples of preferred natural polymers include proteins such as albumin, collagen, gelatin and prolamines, for example, zein, and polysaccharides such as alginate, chitosan, cellulose, carboxymethyl cellulose (CMC), cellulose derivatives, and polyhydroxyalkanoates, for example, polyhydroxybutyrate. The in vivo stability of the particles can be adjusted during the production by using polymers such as poly(lactide-co-glycolide) copolymerized with polyethylene glycol (PEG). If PEG is exposed on the external surface, it may increase the time these materials circulate due to the hydrophilicity of PEG.

Examples of preferred non-biodegradable polymers include ethylene vinyl acetate, poly(meth)acrylic acid, polyamides, copolymers and mixtures thereof. Examples of preferred biodegradable polymers include polyester or polyester segments poly(lactic acid) (PLA), poly(glycolic acid) PGA, or poly(lactide-co-glycolide) (PLGA).

2. Lipid-Based Nanoparticles

Liposomes are spherical vesicles, composed of concentric phospholipid bilayers separated by aqueous compartments. Liposomes adhere to and create a molecular film on cellular surfaces. The lipid vesicles comprise either one or several aqueous compartments delineated by either one (unilamellar) or several (multilamellar) phospholipid bilayers. Liposomes have been widely studied as drug carriers for a variety of chemotherapeutic agents (thousands of scientific articles have been published on the subject).

Liposomes contain one or more lipids. The lipids can be neutral, anionic or cationic lipids at physiologic pH. Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including, but limited to, 1,2-diacyl-glycero-3-phosphocholines; phosphatidylserine (PS), phosphatidylglycerol, phosphatidylinositol (PI), glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids. In one embodiment, the liposomes contain a phosphatidylcholine (PC) head group, and preferably sphingomyelin. In a preferred embodiment, the liposomes contain DPPC. In a preferred embodiment, the liposomes contain a neutral lipid, preferably 1,2-dioleoylphosphatidylcholine (DOPC).

The liposomes typically have an aqueous core. The aqueous core can contain water or a mixture of water and alcohol. Suitable alcohols include, but are not limited to, methanol, ethanol, propanol (such as isopropanol), butanol (such as n-butanol, isobutanol, sec-butanol, tert-butanol), pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

3. Dendrimeric Particles

The term “dendrimer” as used herein includes, but is not limited to, a molecular architecture with an interior core, interior layers (or “generations”) of repeating units regularly attached to this initiator core, and an exterior surface of terminal groups attached to the outermost generation. Examples of dendrimers include, but are not limited to, PAMAM, polyester, polylysine, and PPI. The PAMAM dendrimers can have carboxylic, amine and hydroxyl terminations and can be any generation of dendrimers including, but not limited to, generation 1 PAMAM dendrimers, generation 2 PAMAM dendrimers, generation 3 PAMAM dendrimers, generation 4 PAMAM dendrimers, generation 5 PAMAM dendrimers, generation 6 PAMAM dendrimers, generation 7 PAMAM dendrimers, generation 8 PAMAM dendrimers, generation 9 PAMAM dendrimers, or generation 10 PAMAM dendrimers. Dendrimers suitable for use include, but are not limited to, polyamidoamine (PAMAM), polypropylamine (POPAM), polyethylenimine, polylysine, polyester, iptycene, aliphatic poly(ether), and/or aromatic polyether dendrimers. Each dendrimer of the dendrimer complex may be of similar or different chemical nature than the other dendrimers (e.g., the first dendrimer may include a PAMAM dendrimer, while the second dendrimer may comprise a POPAM dendrimer). In some embodiments, the first or second dendrimer may further include an additional agent. The multiarm PEG polymer includes a polyethylene glycol having at least two branches bearing sulfhydryl or thiopyridine terminal groups; however, embodiments are not limited to this class and PEG polymers bearing other terminal groups such as succinimidyl or maleimide terminations can be used. The PEG polymers in the molecular weight 10 kDa to 80 kDa can be used.

A dendrimer complex includes multiple dendrimers. For example, the dendrimer complex can include a third dendrimer; wherein the third-dendrimer is complexed with at least one other dendrimer. Further, a third agent can be complexed with the third dendrimer. In another embodiment, the first and second dendrimers are each complexed to a third dendrimer, wherein the first and second dendrimers are PAMAM dendrimers and the third dendrimer is a POPAM dendrimer. Additional dendrimers can be incorporated without departing from the spirit of the invention. When multiple dendrimers are utilized, multiple agents can also be incorporated. This is not limited by the number of dendrimers complexed to one another.

As used herein, the term “PAMAM dendrimer” means poly(amidoamine) dendrimer, which may contain different cores, with amidoamine building blocks. The method for making them is known to those of skill in the art and generally, involves a two-step iterative reaction sequence that produces concentric shells (generations) of dendritic 6-alanine units around a central initiator core. This PAMAM core-shell architecture grows linearly in diameter as a function of added shells (generations). Meanwhile, the surface groups amplify exponentially at each generation according to dendritic-branching mathematics. They are available in generations G0-10 with 5 different core types and 10 functional surface groups. The dendrimer-branched polymer may consist of polyamidoamine (PAMAM), polyglycerol, polyester, polyether, polylysine, or polyethylene glycol (PEG), polypeptide dendrimers. Dendrimers are also ideal amphiphilic surfactants that have been applied in multiple applications that include bile salts. The aggregates of dendrimers and bile salts are also a kind of mixed micelles that have distinct properties compared to traditional surfactants with hydrophilic head and hydrophobic tails. In some embodiments, the dendrimers are in nanoparticle form as described in WO2009/046446.

4. Methods of Making Particles

Common techniques for preparing nanoparticles include, but are not limited to, solvent evaporation, solvent removal, self-assembly, spray drying, phase inversion, coacervation, and low temperature casting. Suitable methods of particle formulation are briefly described below. Pharmaceutically acceptable excipients, including pH modifying agents, disintegrants, preservatives, and antioxidants, can optionally be incorporated into the particles during particle formation.

A. Solvent Evaporation

In this method, the drug (or polymer matrix and one or more Drugs) is dissolved in a volatile organic solvent, such as methylene chloride. The organic solution containing the drug is then suspended in an aqueous solution that contains a surface active agent such as poly(vinyl alcohol). The resulting emulsion is stirred until most of the organic solvent evaporated, leaving solid nanoparticles. The resulting nanoparticles are washed with water and dried overnight in a lyophilizer. Nanoparticles with different sizes and morphologies can be obtained by this method.

Drugs which contain labile polymers, such as certain polyanhydrides, may degrade during the fabrication process due to the presence of water. For these polymers, the following two methods, which are performed in completely anhydrous organic solvents, can be used.

B. Solvent Removal

Solvent removal can also be used to prepare particles from drugs that are hydrolytically unstable. In this method, the drug (or polymer matrix and one or more Drugs) is dispersed or dissolved in a volatile organic solvent such as methylene chloride. This mixture is then suspended by stirring in an organic oil (such as silicon oil) to form an emulsion. Solid particles form from the emulsion, which can subsequently be isolated from the supernatant. The external morphology of spheres produced with this technique is highly dependent on the identity of the drug.

C. Spray Drying

In this method, the drug (or polymer matrix and one or more Drugs) is dissolved in an organic solvent such as methylene chloride. The solution is pumped through a micronizing nozzle driven by a flow of compressed gas, and the resulting aerosol is suspended in a heated cyclone of air, allowing the solvent to evaporate from the microdroplets, forming particles. Particles ranging between 0.1-10 microns can be obtained using this method.

D. Phase Inversion

Particles can be formed from drugs using a phase inversion method. In this method, the drug (or polymer matrix and one or more Drugs) is dissolved in a “good” solvent, and the solution is poured into a strong non solvent for the drug to spontaneously produce, under favorable conditions, microparticles or nanoparticles. The method can be used to produce nanoparticles in a wide range of sizes, including, for example, about 100 nanometers to about 10 microns, typically possessing a narrow particle size distribution.

E. Coacervation

Techniques for particle formation using coacervation are known in the art, for example, in GB-B-929 406; GB-B-929 40 1; and U.S. Pat. Nos. 3,266,987, 4,794,000, and 4,460,563. Coacervation involves the separation of a drug (or polymer matrix and one or more Drugs) solution into two immiscible liquid phases. One phase is a dense coacervate phase, which contains a high concentration of the drug, while the second phase contains a low concentration of the drug. Within the dense coacervate phase, the drug forms nanoscale or microscale droplets, which harden into particles. Coacervation may be induced by a temperature change, addition of a non-solvent or addition of a micro-salt (simple coacervation), or by the addition of another polymer thereby forming an interpolymer complex (complex coacervation).

F. Self-Assembly

Numerous methods and materials have been used to form nanoparticles for self-assembly. for example, Gu, et al., describes formation of PLGA-PEG/PLGA blended nanoparticles by self-assembly. See Proc Natl Acad Sci USA. 2008 Feb. 19; 105(7): 2586-2591. NPs were formulated by the self-assembly of an amphiphilic triblock copolymer composed of end-to-end linkage of poly(lactic-co-glycolic-acid) (PLGA), polyethyleneglycol (PEG), and an active agent.

PEG-PLGA block copolymers can be used to prepare particles. Although various kinds of block copolymers can be synthesized, the most commonly synthesized block copolymers have AB, BAB, or ABA block structures, where A and B stand for PEG and PLGA blocks, respectively. Synthetic methods for producing these copolymers are well established and a number of block copolymers are commercially available from companies such as Akina (http://www.akinainc.com) and Polysciences, Inc (http://www.polysciences.com). Block copolymers with low molecular weights and/or high PEG/PLGA ratios are water-soluble, whereas those with high molecular weights and/or low PEG/PLGA ratios are water-insoluble. Block copolymers, which are more hydrophilic than bare PLGA, are considered to be more suitable for the delivery of hydrophilic macromolecules such as proteins.

Due to their amphiphilic nature, when PEG-PLGA block copolymers are dispersed in an aqueous medium, they self-assemble into micellar forms. PEG acts as a hydrophilic corona, while PLGA serves as a hydrophobic core. A polymeric micelle can incorporate aqueous hydrophobic drugs such as paclitaxel. Polymeric micelles can prolong the blood residence time of drugs, lessen systemic toxicity, and direct drugs to the site of action

Nanoprecipitation is another method for nanoparticles preparation. The self-assembly feature of poly (ethylene glycol)-poly (lactide-co-glycolic acid) (PEG-PLGA) amphiphilic copolymer into a nanoparticle and its versatile structure makes nanoprecipitation one of the best methods for its preparation.

### IV. Targeting Agents

The nanoparticles of the present disclosure may be combined with at least one targeting agent. In some embodiments, the targeting agent is directed to CD2. In some embodiments, the targeting agent is directed to CD3. In some embodiments, more than one targeting agent may be used. In some embodiments, the targeting agent is directed to CD2 and CD3. In some embodiments, the targeting agent is directed to T cells. In some embodiments, the targeting agent is directed to NK cells. In some embodiments, the targeting agent targets T cells and NK cells. In some embodiments, the targeting agent targets NKT cells. In some embodiments the targeting agent directed to T cells targets a receptor on the surface of T cells. In some embodiments the targeting agent directed to NK cells targets a receptor on the surface of NK cells. In some embodiments, the at least one targeting agent is at least one member selected from a group consisting of: an anti-CD2 antibody, an anti-CD3 antibody, and an anti-CD3 antibody with an inactivated or absent Fc fragment. Anti-CD2 and anti-CD3 can also target NKT cells. In some embodiments, targeting CD3 induces NKT cells to become T regulatory cells. In some embodiments, targeting CD3 induces NKT cells to become Foxp3+ T regulatory cells.

The nanoparticles are targeted to CD2 that is expressed on T cells and NK cells, or targeted to CD3 which is expressed on T cells. Both CD2 and CD3 are signaling receptors that induce lymphocyte activation and can influence differentiation. Tregs require stimulation for induction and continuous stimulation for function and survival. A targeting moiety may be a nucleic acid (e.g. aptamer), polypeptide (e.g. antibody), glycoprotein, small molecule, carbohydrate, lipid, etc. For example, a targeting moiety can be an aptamer, which is generally an oligonucleotide (e.g., DNA, RNA, or an analog or derivative thereof) that binds to a particular target, such as a polypeptide. In general, the targeting function of the aptamer is based on the three-dimensional structure of the aptamer. In some embodiments, a targeting moiety is a polypeptide such as an antibody or antibody fragment.

In one preferred embodiment the particles are targeted to natural killer (“NK”) cells which express CD2. In another preferred embodiment the particles are targeted to T cells which express CD3. Typically, the targeting molecules exploit the surface-markers specific to a biologically functional class of cells, such as T cells. For example, T cells express a number of cell surface markers, such as CD2 which is a transmembrane molecule and a member of the immunoglobulin supergene family that plays an important role in T-cell activation, T- or NK-mediated cytolysis, apoptosis in activated peripheral T cells, and the production of cytokines by T cells. CD3 is the signaling component of the T cell receptor which recognize peptide/MHC complexes expressed by antigen-presenting cells. Targeting molecules may result in internalization of the nanoparticle or other delivery vehicle within the target cell or tissue. For example, in some embodiments, the nanoparticle or other delivery vehicle can be targeted to a cell surface receptor that can mediate endocytosis. Accordingly, in some embodiments, the nanoparticles can be targeted via lectin-mediated endocytosis.

In some embodiments, the targeting agents, in addition to being capable of specifically binding to a target, also act as stimulating agents. For example, targeting CD2 or CD3 can stimulate the production of TGF-β. In some embodiments, targeting CD3 may stimulate proliferation and/or differentiation of a cell that expresses CD3 on the cell surface.

A. Antibodies

In some embodiments, nanoparticles are modified to include one or more antibodies. Antibodies that function by binding directly to one or more epitopes, other ligands, or accessory molecules at the surface of cells can be coupled directly or indirectly to the nanoparticles. In some embodiments, the antibody or antigen binding fragment thereof has affinity for a receptor at the surface of a specific cell type, such as a receptor expressed on the surface of T cells. The antibody may bind one or more target receptors at the surface of a cell that enables, enhances or otherwise mediates cellular uptake of the antibody-bound nanoparticle, or intracellular translocation of the antibody-bound nanoparticle, or both.

Any specific antibody can be used to modify the nanoparticles. For example, antibodies can include an antigen binding site that binds to an epitope on the target cell. Binding of an antibody to a “target” cell can enhance or induce uptake of the associated nanoparticle by the target cell protein via one or more distinct mechanisms.

In some embodiments, the antibody or antigen binding fragment binds specifically to an epitope. The epitope can be a linear epitope. The epitope can be specific to one cell type or can be expressed by multiple different cell types. The antibody or antigen binding fragment thereof can bind a conformational epitope that includes a 3-D surface feature, shape, or tertiary structure at the surface of the target cell.

Various types of antibodies and antibody fragments can be used to target the nanoparticles, including whole immunoglobulin of any class, fragments thereof, and synthetic proteins containing at least the antigen binding variable domain of an antibody. The antibody can be an IgG antibody, such as IgG1, IgG2, IgG3, or IgG4 subtypes. An antibody can be in the form of an antigen binding fragment including a Fab fragment, F(ab′)2 fragment, a single chain variable region (scFv), diabodies, triabodies and the like.

The antibody can be a naturally occurring antibody, e.g., an antibody isolated and/or purified from a mammal, e.g., mouse, rabbit, goat, horse, chicken, hamster, human, etc. Alternatively, the antibody can be a genetically engineered antibody, e.g., a humanized antibody. Antibodies can be polyclonal, or monoclonal (mAb). Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity (U.S. Pat. No. 4,816,567; and Morrison, et al., Proc. Natl. Acad. Sci. USA, 81: 6851-6855 (1984)). The antibodies can also be modified by recombinant means, for example by deletions, additions, and/or substitutions of amino acids, to increase efficacy of the antibody in mediating the desired function. Substitutions can be conservative substitutions. For example, at least one amino acid in the constant region of the antibody can be replaced with a different residue (see, e.g., U.S. Pat. Nos. 5,624,821; 6,194,551; WO 9958572; and Angel, et al., Mol. Immunol. 30:105-08 (1993)). In some cases changes are made to reduce undesired activities, e.g., complement-dependent cytotoxicity. The antibody can be a bi-specific antibody having binding specificities for at least two different antigenic epitopes. In some embodiments, the epitopes are from the same antigen. In some embodiments, the epitopes are from two different antigens. Bi-specific antibodies can include bi-specific antibody fragments (see, e.g., Hollinger, et al., Proc. Natl. Acad. Sci. U.S.A., 90:6444-48 (1993); Gruber, et al., J. Immunol., 152:5368 (1994)).

In preferred embodiments, the targeting agent is an antibody or antigen binding fragment thereof that recognizes and/or binds to CD3 and/or CD2 expressing cells. CD2 and CD3 antibodies are known in the art (e.g., Abcam catalog no. 1E78.G4 (anti-CD2) ab5690 (anti-CD3) and R&D Systems catalog no. MAB18561 (anti-CD2), MAB100 (anti-CD3). In some embodiments, the targeting agent targets an extracellular portion of CD3 or CD2. The domains of CD3 and CD2 and the nucleic acid or protein sequences corresponding to these domains are known in the art. Nucleic acid and protein sequences for human CD3 and CD2 are known in the art. See, for example, the sequences referenced in Table 1, which are hereby incorporated by reference.

TABLE 1
Uniprot and GenBank Accession Numbers
for CD2 and CD3 sequences

| Cell surface marker | Sequence | Database: Accession No. |
|---|---|---|
| CD2 | mRNA (cDNA) | GenBank: BC033583.1 |
| CD2 | protein | UniProt: P06729 |
| CD3 | mRNA (cDNA) | GenBank: BC025782.1 |
| CD3 | protein | UniProt: P01730 |

Antibodies that target the nanoparticle to a specific epitope (e.g., a particular domain of a target antigen) can be generated by any means known in the art. Exemplary descriptions means for antibody generation and production include Delves, Antibody Production: Essential Techniques (Wiley, 1997); Shephard, et al., Monoclonal Antibodies (Oxford University Press, 2000); Goding, Monoclonal Antibodies: Principles and Practice (Academic Press, 1993); and Current Protocols in Immunology (John Wiley & Sons, most recent edition). Fragments of intact Ig molecules can be generated using methods well known in the art, including enzymatic digestion and recombinant means.

B. Aptamers

In some embodiments, nanoparticles or other delivery vehicles described herein are conjugated with or incorporate aptamers which can contribute to the preferential targeting to one or more types of cells, tissues, organs, or microenvironments. In some embodiments, the aptamer can enhance internalization of the nanoparticle or other delivery vehicle into a cell (e.g., if the aptamer binds to a cell-surface marker).

Aptamers are short single-stranded DNA or RNA oligonucleotides (6˜26 kDa) that fold into well-defined 3D structures that recognize a variety of biological molecules including transmembrane proteins, sugars and nucleic acids with high affinity and specificity (Yu B, et al., Mol Membr Biol., 27(7):286-98 (2010)). The high sequence and conformational diversity of naïve aptamer pools (not yet selected against a target) makes the discovery of target binding aptamers highly likely. Aptamers preferably interact with a target molecule in a specific way. Typically aptamers are small nucleic acids ranging from 15-50 bases in length that fold into defined secondary and tertiary structures, such as stem-loops or G-quartets. Aptamers can bind small molecules, such as ATP and theophylline, as well as large molecules, such as reverse transcriptase and thrombin. Aptamers can bind very tightly to the target molecule, with Kds of less than 10-12 M. It is preferred that the aptamers bind the target molecule with a Kd less than 10-6, 10-8, 10-10, or 10-12. Aptamers can bind the target molecule with a very high degree of specificity. For example, aptamers have been isolated that have greater than a 10,000 fold difference in binding affinities between the target molecule and another molecule that differ at only a single position on the molecule. It is preferred that the aptamer have a Kd with the target molecule at least 10, 100, 1000, 10,000, or 100,000 fold lower than the Kd with a background binding molecule. It is preferred when doing the comparison for a molecule such as a polypeptide, that the background molecule be a different polypeptide. In some embodiments, aptamers binding TCR-CD3 are used in place of the antibodies described herein. Examples of aptamers that may be used in the present disclosure are described in Zumrut H E, et al Ann Biochem 512:1-7, 2016, DOI 10.1016/j and are incorporated by reference herein.

In some embodiments, the aptamer specifically binds to cell surface or transmembrane proteins, such as, but not limited to, CD2 or CD3. In some embodiments, one or more aptamers specifically bind to cell surface and/or transmembrane proteins, such as, but not limited to, CD2 or CD3.

The preferred targeting agent is an antibody, humanized antibody, or antibody fragment thereof having the same binding specificity. These are bound to the surface of the particles, or to the polymers forming the particle, so that the targeting agents appear on the surface of the particles.

Suitable crosslinking agents are disclosed in Tables 1 and 2 below. Other suitable crosslinking agents include avidin, neutravidin, streptavidin, and biotin.

The particles may be functionalized using any suitable chemical modifications of the additives in the continuous matrix. An example is a copper-free click chemistry that can be used to functionalize the surface of the particles to bind any ligand or moiety of interest, including linkers, peptides, antibodies, and fluorescent or radiolabeled reporter molecules.

In preferred embodiments, particles containing a tethering moiety and/or a tethered particle, may have linking moieties on the surface to link the tethering moiety to the core particle, the tethered particle to the core particle, the tethering moiety to the tethered particle, or the tethering moiety and the tethered particle to the core particle. The linking moieties may be proteins, peptides, or small molecules or short polymers. The linking moieties may be crosslinking agents. Crosslinking agents are categorized by their chemical reactivity, spacer length, and materials.

TABLE 2
Reactive groups of crosslinking agents

| Reactivity Class | Chemical Group of Crosslinking |
|---|---|
| (Reactive group) | Agent |
| Carboxyl-to-amine | Carbodiimide (e.g. EDC) |
| Amine | NHS ester, Imidoester, |
| | Pentafluorophenyl ester, |
| | Hydroxymethyl phosphine |
| Sulfhydryl | Maleimide, Haloacetyl (Bromo- or |
| | Iodo-) Pyridyldisulfide, |
| | Thiosulfonate, Vinylsulfone |
| Aldehyde | Hydrazide, Alkoxyamine |
| (i.e. oxidized sugars, carbonyls) | |
| Photoreactive groups | Diazine, Aryl Azide |
| (i.e. nonselective, random | |
| insertion) | |
| Hydroxyl (non-aqueous) | Isocyanate |

TABLE 3
Hetero-bi-functional cross-linkers

| Linker | Reactive Toward | Advantages |
|---|---|---|
| SMPT | Primary amines | Great stability |
| | Sulfhydryls | |
| SPDP | Primary amines | Thiolation |
| | Sulfhydryls | Cleavable cross-linker |
| LC-SPDP | Primary amines | Extended spacer arm |
| | Sulfhydryls | |
| Sulfo-LC- | Primary amines | Extended spacer arm; water |
| SPDP | Sulfhydryls | soluble |
| SMCC | Primary amines | Stable maleimide reactive |
| | Sulfhydryls | group; |
| Sulfo- | Primary amines | Stable maleimide reactive |
| SMCC | Sulfhydryls | group; water soluble |
| MBS | Primary amines | |
| | Sulfhydryls | |
| Sulfo-MBS | Primary amines | Water soluble |
| | Sulfhydryls | |
| SIAB | Primary amines | |
| | Sulfhydryls | |
| Sulfo-SIAB | Primary amines | Water soluble |
| | Sulfhydryls | |
| SMPB | Primary amines | Extended spacer arm |
| | Sulfhydryls | |
| Sulfo- | Primary amines | Extended spacer arm; water |
| SMPB | Sulfhydryls | soluble |
| EDC/Sulfo- | Primary amines | |
| NHS | Carboxyl groups | |
| ABH | Carbohydrates | Reactive with sugar groups |
| | Nonselective | |

### V. Stimulating Agents

Polymeric nanoparticles contain one or more stimulating agents that can induce or increase the expansion and/or function of CD4+ and/or CD8+ Treg cells. The nanoparticles may also induce or increase the population of protective NK cells, for example, in vivo or ex vivo. In some embodiments, the one or more stimulating agents are loaded into or encapsulated within the nanoparticles or directly or indirectly attached (e.g., covalently or non-covalently) to the surface of the nanoparticles for delivery. In preferred embodiments, the stimulating agents are immunomodulatory agents, growth factors or cytokines. In some embodiments, a stimulating agent is IL-2. In some embodiments, a stimulating agent is TGF-β. In some embodiments, stimulating agents are IL-2 and TGF-β. IL-2 and TGF-β are required to induce CD4 and other T cells to become Tregs (Chen W et al. J Exp Med 198:1875-86, 2003). In some embodiments, stimulating agents are therapeutic agents. In some embodiments, stimulating agents are prophylactic agents. In some embodiments, stimulating agents are listed herein under section C as other active agents.

In a preferred embodiment, the nanoparticles contain TGF-β in combination with IL-2. In the most preferred embodiment, IL-2 only is loaded into or encapsulated by the nanoparticles. In the most preferred embodiment, the IL-2 has been modified so that it stimulates Tregs, but not non-Tregs (Spangler J B et al. J Immunol 201:2094-2106, 2018) The nanoparticles targeted to the CD2 and/or CD3 ligands induce the TGF-β required for the induction of the Tregs.

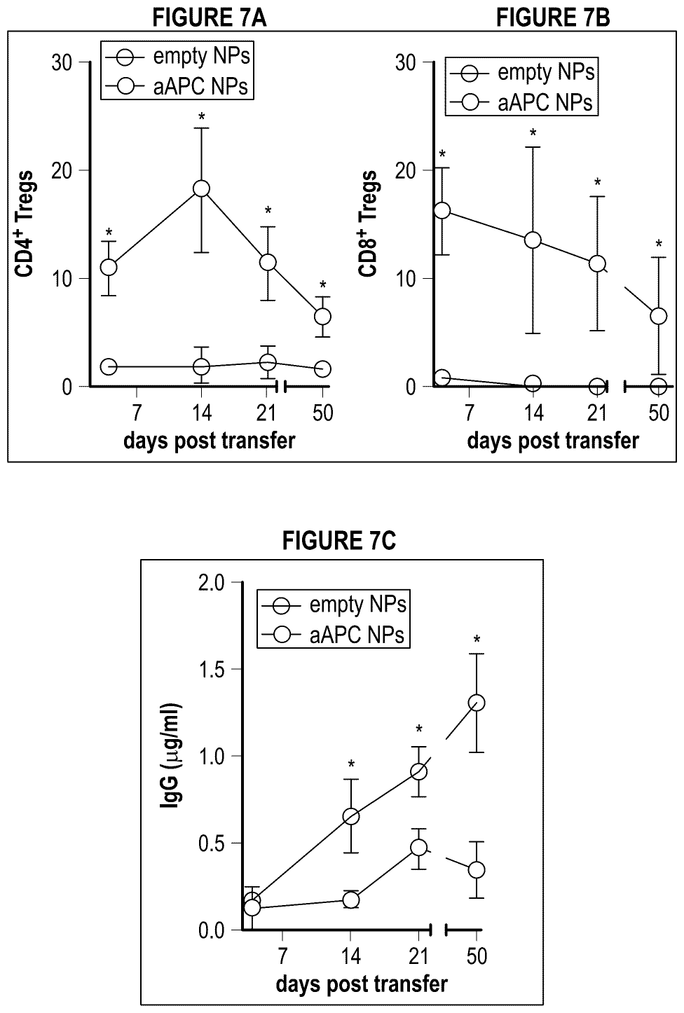

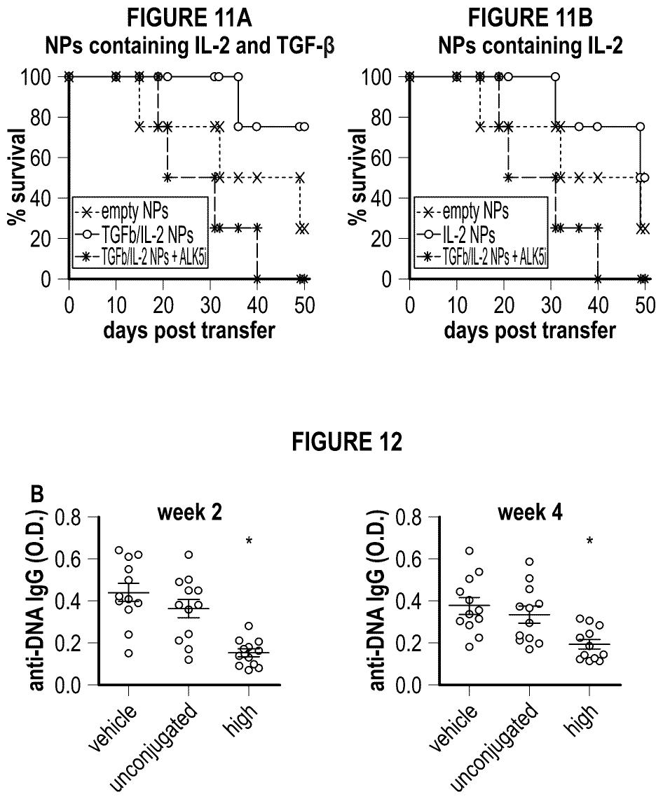

The experiments shown in FIGS. 7 through 12 show aAPCs inducing human Tregs. They indicate that human T cells can also be induced to become suppressive CD4 and CD8 Tregs In Vitro and In Vivo with aAPC NPs.

A. TGF-β

The transforming growth factor beta (TGF-β) superfamily is a family of pleiotropic cytokines that regulates multifaceted cellular functions including proliferation, differentiation, migration, and survival. The TGF-β superfamily is a large and continuously expanded group of regulatory polypeptides, including a model transforming growth factor beta family and other families, such as bone morphogenetic proteins (BMPs), growth and differentiation factors (GDFs), activins (ACTs), inhibins (INHs), and glial-derived neurotrophic factors (GDNFs) (Wan Y. and Flavell R., Immunol. Rev., 220:199-213 (2007)).

The model TGF-β family includes three isoforms: TGF-β1, TGF-β2, and TGF-β3. While sharing similar functions, these isoforms are differentially expressed in a spatially and temporally dependent manner. In the immune system, TGF-β1 is the isoform predominantly expressed. TGF-β is synthesized in an inactive form, pre-pro-TGF-β precursor. Additional stimuli are required to liberate active TGF-β, enabling it to exert its function in either a cell surface-bound form or a soluble form (Wan Y. and Flavell 2007)).

Identified as a growth factor for transformed tumor cells, TGF-β in fact inhibits the proliferation of non-transformed cells, such as epithelial cells and fibroblasts. TGF-β regulates the adaptive immunity components, such as T cells, as well as the innate immunity components, such as natural killer (NK) cells. TGF-β can promote either T-helper 17 cells (Th17) or regulatory T-cells (Treg) lineage differentiation in a concentration-dependent manner. TGF-β suppresses immune responses through two means: inhibiting the function of inflammatory cells and promoting the function of Treg cells (Wan Y. and Flavell R; 2007)). Regarding the latter, TGF-β inhibits immune responses by promoting the generation of Treg cells by inducing Foxp3 expression. Early studies demonstrated that TGF-β was necessary and sufficient for human CD8+ T cells to acquire suppressive activities.

Protein, mRNA, and gene sequences for TGF-β1 are known in the art.

For example, a protein sequence for human TGF-β1 is:

(SEQ ID NO: 1; UniProt ID No. P01137)
MPPSGLRLLLLLLPLLWLLVLTPGRPAAGLSTCKTIDMELVKRKRIEAI
RGQILSKLRLASPPSQGEVPPGPLPEAVLALYNSTRDRVAGESAEPEPE
PEADYYAKEVTRVLMVETHNEIYDKFKQSTHSIYMFFNTSELREAVPEP
VLLSRAELRLLRLKLKVEQHVELYQKYSNNSWRYLSNRLLAPSDSPEWL
SFDVTGVVRQWLSRGGEIEGFRLSAHCSCDSRDNTLQVDINGFTTGRRG
DLATIHGMNRPFLLLMATPLERAQHLQSSRHRRALDTNYCFSSTEKNCC
VRQLYIDFRKDLGWKWIHEPKGYHANFCLGPCPYIWSLDTQYSKVLALY
NQHNPGASAAPCCVPQALEPLPIVYYVGRKPKVEQLSNMIVRSCKCS.

An exemplary mRNA sequence (provided as cDNA) for human TGF-β1 is:

ATGCCGCCCTCCGGGCTGCGGCTGCTGCTGCTGCTGCTACCGCTGCTGT
GGCTACTGGTGCTGACGCCTGGCCGGCCGGCCGCGGGACTATCCACCTG
CAAGACTATCGACATGGAGCTGGTGAAGCGGAAGCGCATCGAGGCCATC
CGCGGCCAGATCCTGTCCAAGCTGCGGCTCGCCAGCCCCCCGAGCCAGG
GGGAGGTGCCGCCCGGCCCGCTGCCCGAGGCCGTGCTCGCCCTGTACAA
CAGCACCCGCGACCGGGTGGCCGGGGAGAGTGCAGAACCGGAGCCCGAG
CCTGAGGCCGACTACTACGCCAAGGAGGTCACCCGCGTGCTAATGGTGG
AAACCCACAACGAAATCTATGACAAGTTCAAGCAGAGTACACACAGCAT
ATATATGTTCTTCAACACATCAGAGCTCCGAGAAGCGGTACCTGAACCC
GTGTTGCTCTCCCGGGCAGAGCTGCGTCTGCTGAGGCTCAAGTTAAAAG
TGGAGCAGCACGTGGAGCTGTACCAGAAATACAGCAACAATTCCTGGCG
ATACCTCAGCAACCGGCTGCTGGCACCCAGCGACTCGCCAGAGTGGTTA
TCTTTTGATGTCACCGGAGTTGTGCGGCAGTGGTTGAGCCGTGGAGGGG
AAATTGAGGGCTTTCGCCTTAGCGCCCACTGCTCCTGTGACAGCAGGGA
TAACACACTGCAAGTGGACATCAACGGGTTCACTACCGGCCGCCGAGGT
GACCTGGCCACCATTCATGGCATGAACCGGCCTTTCCTGCTTCTCATGG
CCACCCCGCTGGAGAGGGCCCAGCATCTGCAAAGCTCCCGGCACCGCCG
AGCCCTGGACACCAACTATTGCTTCAGCTCCACGGAGAAGAACTGCTGC
GTGCGGCAGCTGTACATTGACTTCCGCAAGGACCTCGGCTGGAAGTGGA
TCCACGAGCCCAAGGGCTACCATGCCAACTTCTGCCTCGGGCCCTGCCC
CTACATTTGGAGCCTGGACACGCAGTACAGCAAGGTCCTGGCCCTGTAC
AACCAGCATAACCCGGGCGCCTCGGCGGCGCCGTGCTGCGTGCCGCAGG
CGCTGGAGCCGCTGCCCATCGTGTACTACGTGGGCCGCAAGCCCAAGGT
GGAGCAGCTGTCCAACATGATCGTGCGCTCCTGCAAGTGCAGCTGA
(SEQ ID NO: 2; GenBank: BC000125.1; Homo sapiens
TGFB1 mRNA, complete cds).

A gene sequence for TGF-β1 can be found as part of the DNA sequence on human chromosome 19q13.2, NCBI Reference Sequence: NG_013364.1. Similar to TGF-β1, protein, mRNA, and gene sequences for TGF-β2 and TGF-β3 are known in the art. Any of the above sequences and variants (e.g., naturally occurring variants), analogs, or derivatives thereof for TGF-β1, as well as variants (e.g., naturally occurring variants), analogs, or derivatives of TGF-β2 and TGF-β3 can be used in providing TGF-β molecules in accordance with the compositions, formulations and methods. In addition, recombinant human TGF-β proteins are commercially available from multiple vendors. For example, recombinant human TGF-β1 is available from Peprotech (catalog no.: 100-21) and Abcam (catalog no.: ab50036).

B. IL-2

In preferred embodiments, the nanoparticles contain IL-2 in combination with TGF-β. Interleukin-2 (IL-2) plays a crucial role in regulating immune responses and maintaining peripheral self-tolerance by having both immuno-stimulatory and immunoregulatory functions. IL-2 signals influence various lymphocyte subsets during differentiation, immune responses and homeostasis. IL-2 acts primarily as a T cell growth factor, essential for the proliferation and survival of T cells as well as the generation of effector and memory T cells. For example, stimulation with IL-2 is crucial for the maintenance of regulatory T (TReg) cells and for the differentiation of CD4+ T cells into defined effector T cell subsets following antigen-mediated activation.

IL-2 is a 15-16 KDa, four α-helix bundle cytokine that belongs to a family of structurally related cytokines that includes IL-4, IL-7, IL-9, IL-15, and IL-21. The IL-2 cytokine displays multiple immunological effects and acts by binding to various forms of the IL-2 receptor (IL-2R), notably the monomeric, dimeric, and trimeric forms. The association of IL-2Rα (CD25), IL-2Rβ (CD122), and IL-2Rγ (CD132) subunits results in the trimeric high affinity IL-2Rαβγ. CD25 confers high affinity binding to IL-2, whereas the β and γ subunits (expressed on natural killer (NK) cells, monocytes, macrophages and resting CD4+ and CD8+ T cells) mediate signal transduction. It appears that the expression of CD25 is essential for the expansion of immunosuppressive regulatory T cells (Treg); on the other hand, cytolytic CD8+ T and NK cells can proliferate and kill target cells responding to IL-2 by the IL-2Rβγ engagement in the absence of CD25 (Mortara L., et al., Front. Immunol., 9:2905 (2018)). Interaction of IL-2 with monomeric IL-2R (IL-2Rα (CD25)) does not induce a signal, but both dimeric (IL-2Rβ (CD122) and IL-2Rγ (CD132)) and trimeric (IL-2Rαβγ) IL-2Rs lead to a downstream signal upon binding to IL-2 (Arenas-Ramirez N., et al., Trends Immunol., 36(12):763-777 (2015)). Regulatory T cells can efficiently respond to IL-2 through the IL-2Rαβγ complex (Mortara L., et al., 2018).

On triggering of IL-2R, IL-2 mediated signal transduction occurs via three major pathways, involving: (i) Janus kinase (JAK)-signal transducer and activator of transcription (STAT); (ii) phosphoinositide 3-kinase (PI3K)-AKT; and (iii) mitogen-activated protein kinase (MAPK) (Arenas-Ramirez N., et al., 2015).

IL-2 can stimulate Treg even at low doses (e.g., 1.5×10⁶-3×10⁶ IU once daily in humans. Low-dose IL-2 has been proposed to be suitable for the treatment of autoimmune and chronic inflammatory diseases such as systemic lupus erythematosus (SLE), type 1 diabetes, and cryoglobulinemic vasculitis, as well as graft rejection and chronic graft-versus-host disease, as these conditions have been reported to often feature lower IL-2 signaling and a relative Treg to effector T cell deficiency (Arenas-Ramirez N., et al., 2015). Conversely, high-dose IL-2 (e.g., 6×10⁵-7.2×10⁵ IU/kg body weight three times daily for up to 14 doses per cycle in humans) has been used for immunotherapy against metastatic cancer as high doses of IL-2 stimulate antitumor cytotoxic lymphocytes, including effector T and NK cells (Arenas-Ramirez N., et al., 2015). To avoid the possibility that IL-2 could stimulate T effector cells, IL-2 has been modified so that it only stimulates Tregs (Spangler J B et al. J Immunol 201:2094-2106, 2018).

Protein, mRNA, and gene sequences for IL-2 are known in the art.

For example, a protein sequence for human IL-2 is:

(SEQ ID NO: 3; UniProt ID No. P60568)
MYRMQLLSCIALSLALVTNSAPTSSSTKKTQLQLEHLLLDLQMILNGIN
NYKNPKLTRMLTFKFYMPKKATELKHLQCLEEELKPLEEVLNLAQSKNF
HLRPRDLISNINVIVLELKGSETTFMCEYADETATIVEFLNRWITFCQS
IISTLT.

An exemplary mRNA sequence (provided as cDNA) for human IL-2 is:

ATGTACAGGATGCAACTCCTGTCTTGCATTGCACTAAGTCTTGCACTTG
TCACAAACAGTGCACCTACTTCAAGTTCTACAAAGAAAACACAGCTACA
ACTGGAGCATTTACTGCTGGATTTACAGATGATTTTGAATGGAATTAAT
AATTACAAGAATCCCAAACTCACCAGGATGCTCACATTTAAGTTTTACA
TGCCCAAGAAGGCCACAGAACTGAAACATCTTCAGTGTCTAGAAGAAGA
ACTCAAACCTCTGGAGGAAGTGCTAAATTTAGCTCAAAGCAAAAACTTT
CACTTAAGACCCAGGGACTTAATCAGCAATATCAACGTAATAGTTCTGG
AACTAAAGGGATCTGAAACAACATTCATGTGTGAATATGCTGATGAGAC
AGCAACCATTGTAGAATTTCTGAACAGATGGATTACCTTTTGTCAAAGC
ATCATCTCAACACTAACTTGA (SEQ ID NO: 4; GenBank:
S77834.1; Homo sapiens IL-2 mRNA,complete cds).

A gene sequence for IL-2 can be found as part of the DNA sequence on human chromosome 4q27, NCBI Reference Sequence: NG_016779.1.

Any of the above sequences and variants (e.g., naturally occurring variants), analogs, or derivatives thereof, can be used in providing IL-2 molecules in accordance with the compositions, formulations and methods. In addition, recombinant human IL-2 proteins are commercially available from multiple vendors and can be used in accordance with the compositions, formulations and methods. For example, recombinant human IL-2 is available from Peprotech (catalog no.: 200-02) and as PROLEUKIN® (aldesleukin).

C. Other Active Agents

Autoantigen peptides: To increase the specificity of the aAPC to induce antigen specific regulatory cells, pathogenic peptides involved in the pathogenesis of SLE, type 1 diabetes, multiple sclerosis and other autoimmune diseases have been identified. These can be attached or encapsulated into the NPs. Synthetic, biodegradable nanoparticles carrying either protein or peptide antigens and a tolerogenic immunomodulator, rapamycin, to induce durable antigen-specific immune tolerance have been used to treat the mouse model of multiple sclerosis (Maldonado R A et al. Proc Nat Acad Sci 112:156-65, 2015). In SLE these peptides include five critical autoepitopes in apoptotic cell derived nucleosomes are in histone (H) regions, H122-42, H382-105, H3115-135, H416-39 and H471-94. These peptides are recognized by autoimmune T and B cells of patients and various mouse strains with SLE and these epitopes are promiscuously bound by all major MHC molecules. (Datta S K Ann NY Acad Sci 987:79-90, 2003). Another peptide recognized by SLE T cells is a constructed artificial peptide (“consensus” peptide [pCONS]) based on an algorithm that defines the T cell stimulatory amino acid sequences from the VH regions of multiple BWF1 IgG antibodies to DNA. (Hahn B H Arthritis Rheum 44:438-441, 2001). In type 1 diabetes single or multiple pathogenic pancreatic peptides include islet cell, insulin, and pro-insulin peptides. These include GAD (glutamic acid decarboxylase) peptides. (Roep B O et al. Lancet 7:65-74, 2019).

Defensins: Defensins are peptidic components of the innate immune system of plants and animals. They can be divided in alpha, beta and theta subgroups. A theta defensin named RTD-1 is a small circular 10 amino acid peptide with exceptionally strong anti-inflammatory and tolerogenic properties that could be loaded or encapsulated into aAPCs (Selsted M E et al, Nature Immunol 6:552-557, 2005).

The compositions (e.g., containing a nanoparticle or other delivery vehicle loaded with TGF-β and/or IL-2) can also include one or more additional agents. The additional agents include, but are not limited to, immunosuppressive agents and anti-inflammatory agents.

The anti-inflammatory agent can be non-steroidal, steroidal, or a combination thereof. Representative steroidal anti-inflammatory agents include but are not limited to glucocorticoids, progestins, mineralocorticoids, corticosteroids, and dexamethasone. Exemplary non-steroidal anti-inflammatory agents include, without limitation, ketorolac, nepafenac, diclofenac, oxicams (such as piroxicam, isoxicam, tenoxicam, sudoxicam) and salicylates (such as aspirin, disalcid, benorylate, trilisate, safapryn, solprin, diflunisal, fendosal).

Immunosuppressive drugs include methotrexate, azathioprine, mycophenylate, and rapamycin.

### VI. Methods of Use

The present disclosure provides methods and compositions for treating and preventing immune-mediated disorders. In some embodiments, methods and compositions described herein prevent an immune-mediated disorder. In some embodiments, methods and compositions described herein treat an immune mediated disorder.

In some embodiments, methods of use include CD2-targeted NPs loaded with IL-2 to induce the expansion of NK cells that suppress lupus-like disease in BDF1 mice through TGF-β-dependent mechanisms.

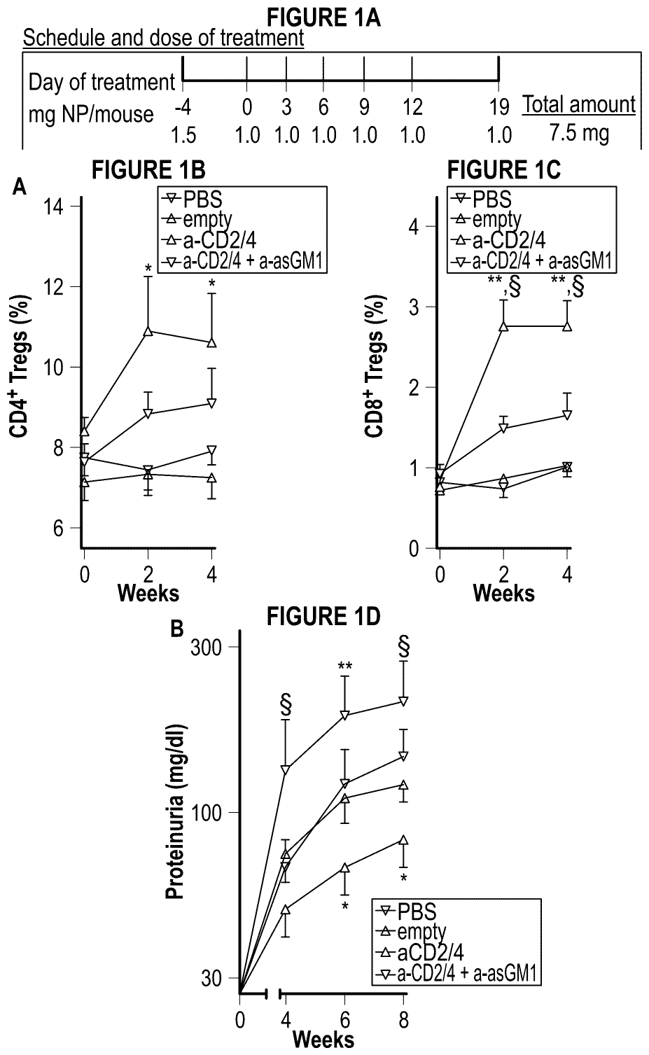

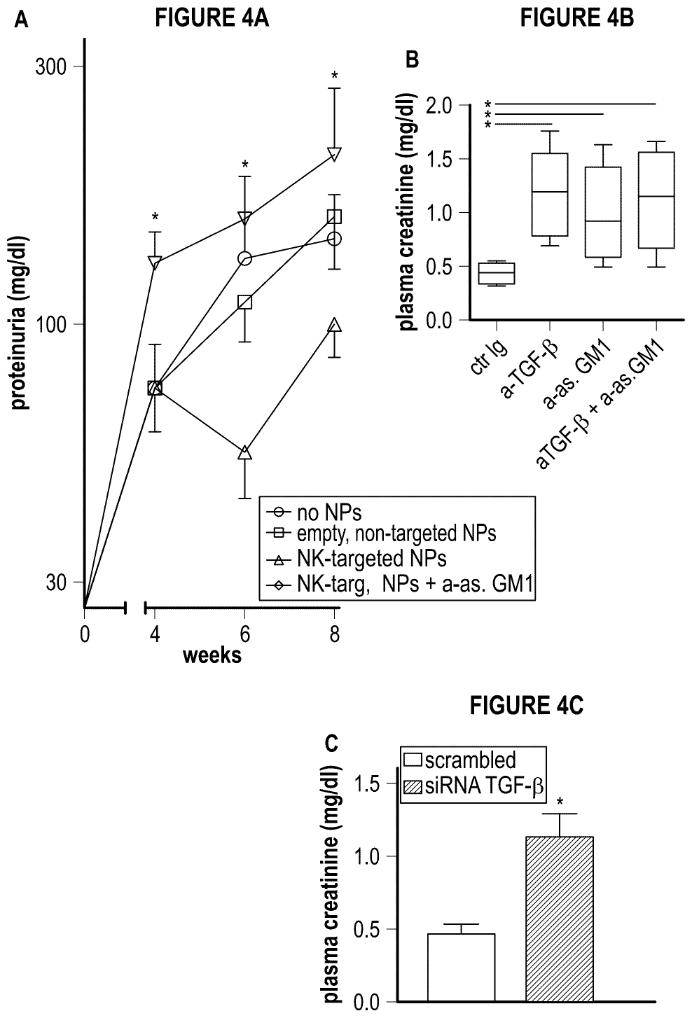

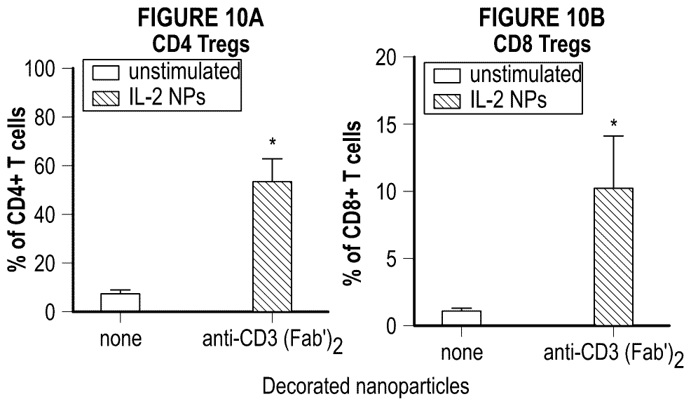

Several strategies have been designed for the suppression of the production of pathogenic autoantibodies in systemic autoimmune diseases. While at present most approaches have been with drugs or biologic agents which have broad effects on both the pathogenic effector cells and the regular cells that control them, our approach has been with methods to selectively induce and expand regulatory cells. In SLE, the use of tolerogenic approaches has generally focused on the induction of immunoregulatory adaptive immune cells, either through ex vivo conditioning of immune cells or through the expansion in vivo of the pool of circulating Tregs. The initial approach used NPs containing tolerogenic cytokines that were coated anti-CD4 and/or CD2 to target T cells in vivo. These NPs induced CD4 and CD8 Tregs that prevented a lupus-like disease in mice. In those studies, we unexpectedly observed involvement of an additional population of immune cells that contributed to the protection from disease. These were NK cells that also express cell surface CD2. When NK cells were depleted from mice that developed a lupus-like disease, the protective effect of our NP aAPCs was abolished. In these mice the expansion of CD4 and CD8 Tregs was suppressed, anti-DNA production was increased and these mice developed more severe renal disease than controls (See FIGS. 1 and 4) On NK cells, CD2 acts synergistically with CD16 for cell activation and this molecule is critical for the control of the antibody response that NK cells can modulate both at the T helper and B cell levels. NK cells are also known to produce cytokines including IFN-γ, TNF-β, GM-CSF and are the principal lymphocyte source of TGF-β (being both the inactive precursor of TGF-β and active TGF-β produced spontaneously by NK cells). After anti-CD2/anti-CD16 antibody stimulation, NK cells produce large amount of TGF-β and IL-10, and anti-CD2 antibodies alone increase TGF-β production—which is decreased in SLE—and promote NK cell-mediated suppression of autoantibody production. Anti-CD2 antibodies induced NK cells to suppress autoantibody production through TGF-β-dependent mechanisms, as indicated by adoptive transfer experiments where the inhibition of TGF-β signaling abolished the NK cell-mediated protective effects (See FIG. 11).

Most studies on CD2 have focused on the expression of this molecule on T cells. For example, the use of anti-CD2 antibodies in autoimmune subjects with multiple sclerosis identified a benign, acute immunosuppression that was only investigated as effects on the recipients' T cells. Similarly, the CD2-specific fusion protein alefacept was found to exert immunosuppressive activity in patients with autoimmune diabetes and the findings were ascribed to a depletion of CD4+ and CD8+ central memory T cells (Tcm) and effector memory T cells (Tem) but an investigation on NK cells was not. Another consideration is that ligation of NK cells by anti-CD2 antibodies could favor the local increase of the concentration of cytokines with long-lasting activity in a bystander recruitment of immune cells. In this context, NPs create a local acidic microenvironment that could favor the conversion of endogenous latent TGF-β to its active form, thus potentiating TGF-β activity even after depletion of its local stores (in the milieu and/or in the NPs).

The compositions and formulations may be prepared as pharmaceutical compositions (e.g., any of the above-described compositions or formulations in combination with a pharmaceutically acceptable buffer, carrier, diluent or excipient) for use in the methods of inducing differentiation of naïve CD4 cells to Tregs ex vivo or in vivo, methods of inducing or increasing the expansion and/or function of CD4+ and/or CD8+ Treg cells ex vivo or in vivo, and/or methods of inducing or increasing a population of NK cells.

The compositions and formulations can be used for therapeutic immunosuppression strategies useful in the treatment of inflammatory diseases or disorders, autoimmune diseases or disorders, inducing or increase graft tolerance, treating graft rejection, and treating allergies and other ailments with symptoms that can be reduced or ameliorated by regulating the activity of T cells, NK cells, antigen-presenting cells, or combinations thereof. In some embodiments, the methods can reduce anti-DNA antibody (e.g., anti-dsDNA autoantibodies) production or reduce renal disease in a subject administered with the compositions.

The method of treatment can include administering to a subject (e.g., a human patient) an effective amount of a pharmaceutical composition containing a nanoparticle that delivers one or more agents (e.g., IL-2, TGF-β) to one or more targeted cells or tissues in the subject. For example, a subject having an autoimmune disease or disorder (e.g., SLE) can be treated by administering to the subject an effective amount of a pharmaceutical composition containing nanoparticles that deliver IL-2 and TGF-β and are targeted with anti-CD2 and/or anti-CD3 antibodies or antigen binding fragments thereof.

The methods initially involved CD2- and/or CD4-targeted nanoparticles (or another delivery vehicle) loaded with TGF-β, IL-2, and optionally one or more other agents, to deliver the agents into cells, or to a cell's microenvironment. Most recently, the methods involve CD2 and/or CD3 targeted nanoparticles. The methods typically include contacting the agent-loaded particles with one or more cells. This contacting can occur in vivo or ex vivo. When used in methods of treatment, the compositions and formulations can be administered to a subject therapeutically or prophylactically.

In some embodiments, the methods and compositions described herein induce lymphocytes in the patient to become multiple populations of functional regulatory cells. In some embodiments, the methods and compositions described herein induce lymphocytes in the patient to become Foxp3+ T regulatory cells. In some embodiments, the methods and compositions described herein induce lymphocytes in the patient to become non-Foxp3+ T regulatory cells. In some embodiments, the methods and compositions described herein induce CD4 and CD8 cells in the patient to become Foxp3+ T regulatory cells In some embodiments, the methods and compositions described herein induce CD4 and CD8 cells in the patient to become non-Foxp3+ T regulatory cells In some embodiments, the methods and compositions described herein induce NK cells in the patient to become non-Foxp3+ T regulatory cells. In some embodiments, the methods and compositions described herein induce NKT cells in the patient to become Foxp3+ T regulatory cells. Examples of non-Foxp3+ T regulatory cells include Tr1 cells that produce IL-10 and TGF-β and Treg3 cells that only produce TGF-β. In some embodiments, the methods and compositions described herein generate and expand regulatory NK cells to numbers that suppress the immune-mediated disorder. In some embodiments, the methods and compositions described herein induce the NK cells in the patient become TGF-β producing regulatory NK cells. In some embodiments, the methods and compositions described herein induce the T cells to become TGF-β producing regulatory T cells. In some embodiments, the methods and compositions described herein reduce numbers of NKT cells. In some embodiments, the methods and compositions described herein reduce numbers of T helper cells. In some embodiments, the methods and compositions described herein reduce numbers of Th1 cells. In some embodiments, the methods and compositions described herein reduce numbers of Th2 cells. In some embodiments, the methods and compositions described herein reduce numbers of Th17 cells. In some embodiments, the methods and compositions described herein reduce numbers of FTH (follicular T helper) cells. In some embodiments, the methods and compositions described herein reduce the function of T helper cells. In some embodiments, the methods and compositions described herein reduce the function of Th1 cells. In some embodiments, the methods and compositions described herein reduce the function Th2 cells. In some embodiments, the methods and compositions described herein reduce the function of Th17 cells. In some embodiments, the methods and compositions described herein reduce the function of FTH (follicular T helper) cells. In some embodiments, the methods and compositions described herein reduce the production of IgG. In some embodiments, the methods and compositions described herein reduce IgG levels. In some embodiments, the methods and compositions described herein reduce the production of autoantibodies. In some embodiments, the methods and compositions described herein reduce autoantibody levels. Examples of assays used to measure changes in cell function and/or phenotype include but are not limited to flow cytometry, CYTOF mass cytometry, ELISA, and DNA or RNA analysis. These and other assays may be used to determine alteration in cell type or function upon treatment with CD2 and/or CD3-targeted nanoparticles loaded with TGF-β and/or IL-2 and/or optionally one or more other agents.

A. Prevention and Treatment of Immune-Mediated Disorders

The subject to be treated may have an immune-mediated disorder, or condition. Some examples of immune-mediated disorders include but are not limited to, diabetes, an immune system disorder such as an autoimmune disease, an inflammatory disease, graft-versus-host disease (GVHD), one or more allergies, or combinations thereof. Therefore, the compositions and methods can be used to treat one or more symptoms of diabetes, an immune system disorder such as an autoimmune disease, an inflammatory disease, graft-versus-host disease (GVHD), one or more allergies, or combinations thereof. In some embodiments, the immune-mediated disorder is an autoimmune disease. In some embodiments, the compositions and methods can be used to treat autoimmune diseases that are antibody-mediated disorders. Autoimmune diseases that are antibody-mediated disorders include, but are not limited to systemic lupus erythematosus, pemphigus vulgaris, myasthenia gravis, hemolytic anemia, thrombocytopenia purpura, Graves disease, dermatomyositis and Sjogren's disease. In some embodiments, the immune-mediated disorder is a cell-mediated autoimmune disorder. Examples of cell-mediated autoimmune disorders include, but are not limited to: type 1 Diabetes, Hashimoto's Disease, polymyositis, inflammatory bowel disease, multiple sclerosis, rheumatoid arthritis and scleroderma. In some embodiments, the immune-mediated disorder is a graft-related disease. In some embodiments, the immune-mediated disorder is a graft-versus-host disease (GVHD). In some embodiments, the immune-mediated disorder is rejection of a foreign organ transplant. In some embodiments, the present methods and compositions may be used to treat an immune-mediated disorder. In some embodiments, the present methods and compositions are administered therapeutically. In some embodiments, the present methods and compositions may be used to prevent an immune-mediated disorder. In some embodiments, the present methods and compositions are administered prophylactically. It has been determined that in autoimmune diseases such as SLE, Rheumatoid Arthritis and type 1 diabetes autoantibodies appear many years before the onset of clinical disease. In some patients the number and amount of these antibodies predict the clinical onset of disease. Administration of the aAPCs to these patients could prevent the onset of clinical disease.

1. Autoimmune Diseases

In some embodiments, the compositions and methods described herein can be used to treat or prevent autoimmune and inflammatory diseases or disorders.

Exemplary autoimmune/inflammatory diseases or disorders, which can be treated or prevented include, but are not limited to, Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticarial, Axonal & neuronal neuropathy (AMAN), Bab disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inverse), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, Ill, Polymyalgia rheumatic, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and Wegener's granulomatosis (or Granulomatosis with Polyangiitis (GPA)).

a. Type I Diabetes

In some embodiments, the compositions and methods described herein can be used to treat or prevent type I diabetes. In some embodiments, insulin producing cells may be transplanted in a subject, and the subject can then be administered an effective amount of the compositions including one or more agents to reduce or inhibit transplant rejection (e.g., TGF-β and IL-2). In some embodiments, the pancreatic islet antigens can be encapsulated together in the nanoparticles or other delivery vehicle with a tolerogenic agent and can be used to induce tolerance toward the insulin producing cells. Preferably the insulin producing cells are beta cells or islet cells. In some embodiments, the insulin producing cells are recombinant cells engineered to produce insulin.

2. Graft-Related Diseases

In some embodiments, the compositions and methods described herein can be used to treat or prevent graft-related diseases. Examples of graft-related diseases include, but are not limited to, graft versus host disease (GVHD) (e.g., such as may result from bone marrow transplantation), immune disorders associated with graft transplantation rejection, chronic rejection, and tissue or cell allo- or xenografts, including solid organs, skin, islets, muscles, hepatocytes, neurons. The compositions and methods described herein can be used for treating or preventing the acute rejection of an organ graft and for reversing the chronic rejection of an organ graft. In some embodiments, the compositions and methods described herein can be used for treating the acute rejection of an organ graft. In some embodiments, the compositions and methods described herein can be used for preventing the acute rejection of an organ graft. In some embodiments, the compositions and methods described herein can be used for treating the chronic rejection of an organ graft. In some embodiments, the compositions and methods described herein can be used for preventing the chronic rejection of an organ graft. In preferred embodiments, the compositions and methods described herein are useful for treatment of solid organ graft rejection. In preferred embodiments, the compositions and methods described herein are useful for treatment of complications associated with stem cell transplantation. In preferred embodiments, the compositions and methods described herein are useful for prevention of complications associated with stem cell transplantation. In preferred embodiments, the compositions and methods described herein are useful for treatment of complications associated with allogenic hematopoietic stem cell transplantation. In preferred embodiments, the compositions and methods described herein are useful for prevention of complications associated with allogenic hematopoietic stem cell transplantation. GVHD is a major complication associated with allogeneic hematopoietic stem cell transplantation in which functional immune cells in the transplanted marrow recognize the recipient as “foreign” and mount an immunologic attack. In preferred embodiments, the compositions and methods described herein are used for treating or alleviating one or more symptoms of graft versus host disease (GVHD) by administering an effective amount of the composition to a subject in need thereof to alleviate one or more symptoms associated with GVHD. In preferred embodiments, the compositions and methods described herein are used for preventing one or more symptoms of graft versus host disease (GVHD) by administering an effective amount of the composition to a subject in need thereof to alleviate one or more symptoms associated with GVHD.

a. Graft Versus Host Disease

In some embodiments, the compositions and methods described herein can be used to prevent or treat graft-versus host disease. Examples of graft-related diseases include graft versus host disease (GVHD) (e.g., such as may result from bone marrow transplantation). In preferred embodiments, the compositions are used for preventing, treating, or alleviating one or more symptoms of graft versus host disease (GVHD) by administering an effective amount of the composition to a subject in need thereof to alleviate one or more symptoms associated with GVHD. GVHD is an immune condition that occurs in a patient after stem cell transplantation, when immune cells present in donor tissue (the graft) attack the host's own tissues. GVHD is a major complication associated with allogeneic hematopoietic stem cell transplantation in which functional immune cells in the transplanted marrow recognize the recipient as “foreign” and mount an immunologic attack. It can also take place in a blood transfusion under certain circumstances. Symptoms of GVHD include, but are not limited to, skin rash, change in skin color or texture, diarrhea, nausea, abnormal liver function, yellowing of the skin, increased susceptibility to infection, dry, irritated eyes, and sensitive or dry mouth.

b. Graft Rejection

In some embodiments, the compositions and methods described herein can be used to prevent treat or prevent graft rejection. Transplantation of foreign tissues that include and tissue or cell allo- or xenografts such as solid organs, skin, islets, muscles, hepatocytes, neurons require the chronic administration of toxic immunosuppressive drugs to avoid or treat acute and chronic graft rejection. In recent years the combination of allogeneic stem cells and the organ graft can lead to mixed chimerism, tolerance and survival of the graft after discontinuing immunosuppressive drugs (Duran-Struuck R, Sykes M. et al. Transplantation 101:274-83, 2017). Nanoparticles have been used to deliver immunosuppressive drugs at lower doses. In preferred embodiments, the compositions and methods can be used to prevent acute and chronic graft rejection without the toxicity of present agents. These methods provide for the IL-2 and TGF-β to induce and sustain Tregs. Here the nanoparticles will contain both IL-2 and TGF-β. Besides binding to the targeted lymphocytes some of the nanoparticles will be phagocytosed by antigen-presenting cells. The TGF-β encapsulated in the nanoparticles will induce these APCs to become tolerogenic (Kosiewicz M M & Alard P. Immunologic Res. 30:155-70, 2006). The addition of subcutaneous injection of peptide MHC antigens that match the organ donor before and continuously after the graft will provide the T cell receptor stimulation required to sustain the alloantigen-specific Tregs needed to prevent graft rejection and avoid the use of immunosuppressive drugs associated with severe toxic side effects.

B. Effective Amounts

The effective amount or therapeutically effective amount of a pharmaceutical composition can be a dosage sufficient to prevent, treat, inhibit, or alleviate one or more symptoms of a disease or disorder, or to otherwise provide a desired pharmacologic and/or physiologic effect, for example, reducing, inhibiting, or reversing one or more of the underlying pathophysiological mechanisms underlying a disease or disorder such as SLE.

In some embodiments, administration of the pharmaceutical compositions (e.g., containing anti-CD2 or anti-CD3 coated nanoparticles loaded with IL-2+/−TGF-β and IL-2) prevents, treats, or alleviates one or more symptoms of an autoimmune disease or disorder, an inflammatory disease or disorder, or an allergy. As such, the amount administered can be expressed as the amount effective to achieve a desired effect in the recipient. For example, in some embodiments, the amount of the pharmaceutical compositions is effective to prevent, reduce or alleviate rashes, nausea, inflammation, diarrhea, or combinations thereof. In some embodiments, the amount of pharmaceutical compositions is effective to induce differentiation of naïve CD4 cells to Tregs in a subject. In some embodiments, the amount of pharmaceutical compositions is effective to induce or increase the expansion and/or function of CD4+ and/or CD8+ Foxp3+ Treg cells in the subject. In some embodiments, the amount of pharmaceutical compositions is effective to reduce or suppress the production of anti-DNA antibodies (e.g., anti-dsDNA autoantibodies) and/or reduce renal disease. In some embodiments, the methods or compositions described herein reduce one or more symptoms of SLE. For example, the amount of pharmaceutical compositions is effective to prevent, delay, or reduce the severity of proteinuria; reduce the production of anti-nuclear autoantibodies (ANA); reduce abnormal lympho-proliferation; prevent, delay or reduce glomerular nephritis; reduce, prevent or delay elevated blood urea levels; or combinations thereof. These effects are also desirable in the treatment of multiple autoimmune diseases such as psoriasis and rheumatoid arthritis.

The effective amount of the pharmaceutical compositions required will vary from subject to subject, depending on the species, age, weight and general condition of the subject, the severity of the disorder being treated, and its mode of administration. Thus, it is not possible to specify an exact amount for every pharmaceutical composition. However, an appropriate amount can be determined by one of ordinary skill in the art using only routine experimentation given the teachings herein. For example, effective dosages and schedules for administering the pharmaceutical compositions can be determined empirically, and making such determinations is within the skill in the art. In some forms, the dosage ranges for the administration of the compositions are those large enough to effect reduction or alleviation of one or more symptoms of a disease or disorder from which the subject suffers.

The dosage should not be so large as to cause adverse side effects, such as unwanted cross-reactions, anaphylactic reactions, and the like. Generally, the dosage will vary with the age, condition, and sex of the patient, route of administration, whether other drugs are included in the regimen, and the type, stage, and location of the disease to be treated. The dosage can be adjusted by the individual physician in the event of any counter-indications. It will also be appreciated that the effective dosage of the composition used for treatment can increase or decrease over the course of a particular treatment. Changes in dosage can result and become apparent from the results of diagnostic assays.

Dosage can vary, and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill can easily determine optimum dosages, dosing methodologies and repetition rates. Optimum dosages can vary depending on the relative potency of individual pharmaceutical compositions, and can generally be estimated based on EC50s found to be effective in in vitro and in vivo animal models.

Generally, dosage levels of 0.001 to 10 mg/kg of body weight daily are administered to mammals. Generally, for intravenous injection or infusion, dosage may be lower. Generally, the total amount of the nanoparticle-associated active agent administered to an individual will be less than the amount of the unassociated active agent that must be administered for the same desired or intended effect. The optimal dosage and treatment regime for a particular patient can readily be determined by one skilled in the art of medicine by monitoring the patient for signs of disease and adjusting the treatment accordingly. In some embodiments, the unit dosage is in a unit dosage form for intravenous injection. In some embodiments, the total amount of IL-2 in the nanoparticles is less than 1000 times the dose administered by standard (non-nanoparticle) IL-2 parenteral injection. In some embodiments, the unit dosage is in a unit dosage form for oral administration. In some embodiments, the unit dosage is in a unit dosage form for inhalation.

Treatment can be continued for an amount of time sufficient to achieve one or more desired therapeutic goals, for example, a reduction of one or more symptoms of a disease relative to the start of treatment. Treatment can be continued for a desired period of time, and the progression of treatment can be monitored using any means known for monitoring the progression of treatment (e.g., anti-inflammatory treatment) in a patient. In some embodiments, administration is carried out every day of treatment, or every week, or every fraction of a week. In some embodiments, treatment regimens are carried out over the course of up to two, three, four or five days, weeks, or months, or for up to 6 months, or for more than 6 months, for example, up to one year, two years, three years, or up to five years.

The efficacy of administration of a particular dose of the pharmaceutical compositions according to the methods described herein can be determined by evaluating the particular aspects of the medical history, signs, symptoms, and objective laboratory tests that are known to be useful in evaluating the status of a subject in need for the treatment of a disease, disorder and/or condition (e.g., SLE) These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved, (2) the progression of the disease or condition is shown to be stabilized, or slowed, or reversed, or (3) the need for other medications for treating the disease or condition is lessened or obviated, then a particular treatment regimen will be considered efficacious. In some embodiments, efficacy is assessed as a measure of quality of life score at a specific time point (e.g., 1-5 days, weeks or months) following treatment.

C. Modes of Administration

Any of the compositions (e.g., containing anti-CD2 and/or CD3-coated nanoparticles loaded with TGF-β and IL-2, or IL-2 only with one or more additional agents) can be used therapeutically in combination with a pharmaceutically acceptable buffer, carrier, diluent or excipient. The compositions described herein can be conveniently formulated into pharmaceutical compositions composed of one or more of the compounds in association with a pharmaceutically acceptable carrier. See, e.g., Remington's Pharmaceutical Sciences, latest edition, by E.W. Martin Mack Pub. Co., Easton, PA, which discloses typical carriers and conventional methods of preparing pharmaceutical compositions that can be used in conjunction with the preparation of formulations of the therapeutics described herein and which is incorporated by reference herein. These most typically would be standard carriers for administration of compositions to humans. In one aspect, for humans and non-humans, these include solutions such as sterile water, saline, and buffered solutions at physiological pH. Other therapeutics can be administered according to standard procedures used by those skilled in the art.

The pharmaceutical compositions described herein can include, but are not limited to, carriers, thickeners, diluents, buffers, preservatives, surface active agents and the like in addition to the active agent(s) of choice.

Pharmaceutical compositions containing one or more agent-loaded nanoparticles can be administered to the subject in a number of ways depending on whether local or systemic treatment is desired, and on the area to be treated. Thus, for example, a pharmaceutical composition can be administered to a subject vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes.

Parenteral administration, if used, is generally characterized by injection. Injectables can be prepared in conventional forms, either as liquid solutions or suspensions, solid forms suitable for solution or suspension in liquid prior to injection, or as emulsions. Another approach for parenteral administration involves use of a slow release or sustained release system such that a constant dosage is maintained. See, e.g., U.S. Pat. No. 3,610,795, which is incorporated by reference herein. Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., intraocular injection, intra-retinal injection, or sub-retinal injection); subcutaneous injection or deposition including subcutaneous infusion (such as by osmotic pumps); direct application by a catheter or other placement device (e.g., an implant comprising a porous, non-porous, or gelatinous material).

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Administration of the pharmaceutical compositions (e.g., containing anti-CD2 and/or CD3-coated nanoparticles loaded with TGF-β and IL-2 or IL-2 only, optionally with one or more additional agents) can be localized (i.e., to a particular region, physiological system, tissue, organ, or cell type) or systemic.

In some preferred embodiments, the pharmaceutical compositions in the present disclosure are administered by parenteral delivery. In some preferred embodiments, the parenteral delivery is intravenous. In some preferred embodiments, the parenteral delivery is intramuscular. In some preferred embodiments, the parenteral delivery is subcutaneous. In some preferred embodiments, the pharmaceutical compositions in the present disclosure are administered by oral delivery.

D. Combination Therapies

In some embodiments, the compositions and formulations are administered to a subject in need thereof in combination with one or more therapeutic, diagnostic, and/or prophylactic agents. For example, an anti-CD2 and/or anti-CD3 coated nanoparticle loaded with IL-2 or IL-2 and TGF-β can be used to deliver an effective amount of TGF-β and IL-2 in combination with one or more therapeutic, diagnostic, and/or prophylactic agents. Alternatively, anti-CD2 and/or anti-CD3-coated nanoparticle loaded with only IL-2 can produce TGF-β locally in combination with one or more diagnostic and/or prophylactic agents. A preferred embodiment is a combination of a tolerogenic aAPC NP with an anti-inflammatory agent that suppress pro-inflammatory cytokines, metalloproteinases and/or inflammatory macrophages.

The term “combination” or “combined” is used to refer to either concomitant, simultaneous, or sequential administration of two or more agents. Therefore, the combinations can be administered either concomitantly (e.g., as an admixture), separately but simultaneously (e.g., via separate intravenous lines into the same subject), or sequentially (e.g., one of the compounds or agents is given first followed by the second). The additional therapeutic, diagnostic, and/or prophylactic agents can be administered locally or systemically to the subject, or coated or incorporated onto or into a device.

The additional agents can be selected based on the disease or disorder to be treated and include, but are not limited to, antibodies, steroidal and non-steroidal anti-inflammatories, TNF-α blockers, immunosuppressants, cytokines, chemokines, defensins, and/or growth factors. Preferably, the additional therapeutic, diagnostic, and/or prophylactic are agents that increase Treg activity or production.

In some embodiments, the present disclosure provides for combination treatment with defensins. In some embodiments, the present disclosure provides for combination treatment with RTD-1. (Tongaonker P et al. Physical Genomics 51:657-67, 2019). In some embodiments, prebiotics may be encapsulated in the nanoparticles to provide for combination treatments.

In preferred embodiments, the therapeutic, diagnostic, and/or prophylactic agents are selected from agents that are clinically used for the treatment of the disease or disorder from which the subject being treated suffers. For example, to treat one or more symptoms of SLE in a subject in need thereof, the methods provide for combined administration of the compositions (e.g., an anti-CD2 and/or anti-CD3-coated nanoparticle loaded with IL-2 and TGF-β, and one or more agents that are used to treat SLE, such as, aspirin, acetaminophen, ibuprofen, naproxen, indomethacin, nabumetone, celecoxib, corticosteroids, cyclophosphamide, methotrexate, azathioprine, belimumab and antimalarials (e.g., hydroxychloroquine and chloroquine). Alternatively, anti-CD2 and/or anti-CD3-coated nanoparticle loaded with only IL-2 which produce TGF-β locally can be used in combination with one or more of the agents used described above to treat lupus.

### VII. Kits

The compositions described above as well as other materials can be packaged together in any suitable combination as a kit useful for performing, or aiding in the performance of, the methods. It is useful if the components in a given kit are designed and adapted for use together in the method. The kits can include, for example, a dosage supply of the composition. The active agents can be supplied alone (e.g., lyophilized), or in a pharmaceutical composition. The active agents can be in a unit dosage, or in a stock that should be diluted prior to administration. In some embodiments, the kit includes a supply of pharmaceutically acceptable carrier. The kits can also contain articles of manufacture such as structures, machines, devices (e.g., for administration), and the like, and compositions, compounds, materials, and the like for use with the provided pharmaceutical compositions. In preferred embodiments, the kit includes devices for administration of the active agents or compositions, for example, syringes. The kits can include printed instructions for administering the compositions in a use as described above. For example, kits may include one or more dosage units of anti-CD2 and/anti-CD3-coated nanoparticles loaded with IL-2+/−TGF-β, IL-2, one or more additional agents, or combinations thereof, and instructions for use.

The present disclosure will be further understood by reference to the following non-limiting examples. These show that mice that develop a lupus-like disease induced by the transfer of splenocytes from DBA/2 mice into (C57BL/6×DBA/2)F1 (BDF1) had significantly reduced lupus disease manifestations and increased survival when treated with NPs loaded with IL-2 and TGF-β targeted to T cells The protective effects conferred by the NPs could not be ascribed only to T cells but also involve additional immune cells because of the targeting of the NPs to CD2+ cells). The key contributors to the suppression of lupus-like disease in BDF1 mice by the NPs are NK cells and the TGF-β produced by these cells. The studies also demonstrate that NPs containing only IL-2 can be used to induce expansion of NK cells ex vivo, which can then be used to treat patients with autoimmune disease, the NK cells producing TGF-β. In the foregoing examples, anti-CD2 Ab is used to target CD8+ cells because of the effects of CD2 in vivo and the capacity of anti-CD2 Ab to induce Foxp3+ Tregs following CD3 stimulation (Ochando et al., J Immunol, 2005; 174:6993-7005), in line with the notion that anti-CD2 Ab and the CD2-specific fusion protein alefacept have immunosuppressive effects in patients with autoimmune disease (Hafler et al., J Immunol, 1988; 141:131-8; Rigby et al., J Clin Invest, 2015; 125:3285-96).

### VIII. Examples

### Example 1. Targeted Nanoparticle Preparation and Characterization

Materials and Methods: Poly(lactic-co-glycolic acid (PLGA) NPs were prepared as described by McHugh et al., Biomaterials, 2015; 59:172-81). Briefly, 60 mg PLGA (Durect, Cupertino, CA) were dissolved in 3 ml of chloroform in a glass test tube. Dropwise addition of 200 μl of an aqueous solution containing carrier-free 1.25 μg IL-2 with or without 2.5 μg TGF-β(PeproTech, Cranbury, NJ), resulted in a primary emulsion which was sonicated and added dropwise to a continuously vortexed glass test tube containing 4 ml of 4.7% polyvinyl alcohol (PVA) and 0.625 mg/ml avidin-palmitate conjugate. The resulting double emulsion was sonicated in an ice bath before transfer to a beaker containing 200 ml of 0.25% polyvinyl acetate (PVA). Particles were allowed to harden by stirring for 3 hours at room temperature and then washed 3 times by cycles of pelleting at 18,000 g and resuspension in Milli-Q water. The washed NPs were flash-frozen in liquid nitrogen and lyophilized, to enable long-term storage at −20° C. until use. The NP preparations underwent examination for physical properties, encapsulation metrics, and release kinetics. Size was quantified using dynamic light scattering with a Malvern Zetasizer Nano. NPs were found to have a hydrodynamic diameter of 245.3±2.2 nm with a low polydispersity index, indicating a uniform NP population with a relatively tight size distribution. Cytokine encapsulation and release were checked by BD OptEIA ELISA kits after disrupting the NPs in dimethyl sulfoxide (DMSO) and by supernatant analysis. For cell targeting, NPs were diluted in phosphate buffered saline (PBS) and incubated with biotinylated anti-CD2 antibody (clone RM2-5, Thermo Fisher Scientific, Waltham, MA) at a ratio of 5-10 μg to 1 mg NPs 10 min. before use.

The therapeutic effect of targeted delivery of IL-2 and TGF-β to T cells for the induction of Tregs in vivo was initially investigated using NPs targeted to CD4+ T cells by coating NPs with anti-CD4 Ab, and to CD8+ T cells by coating with anti-CD2 Ab due to the reported induction of CD8+ Tregs ex vivo with IL-2 and TGF-β via CD2 (Horwitz, D A et al., Arthritis Rheumatol, 2019; 71:632-640). Although CD4regs have potent suppressive effects, the protective effects f CD8+ Tregs in SLE, both alone and in combination with CD4+ Tregs (Dinesh et al., Autoimmun Rev, 2010; 9:560-8; Hahn et al., J Immunol, 2005; 175:7728-37).

For targeting, NPs were freshly prepared at the target concentration in phosphate buffered saline (PBS) and reacted with the biotinylated targeting antibody at a concentration ratio of 2 μg Ab to 1 mg NPs 10 minutes prior to use. NPs size was quantified using dynamic light scattering (DLS) with a Malvern Zetasizer Nano. Cytokine encapsulation and release were measured by BD OPTEIA™ ELISA kits, either after disrupting particles in DMSO or by supernatant analysis of release study aliquots. For the release assay, a 1 wt/v % solution of PLURONIC F127 in PBS was used as release buffer, to help stabilize released cytokine and prevent binding to the tube surface and loss of capture/detection antibody binding ability. The release assay was performed using 1 mg/ml aliquots of particles in release buffer. At each time point, aliquots were spun down in a microcentrifuge and supernatant was isolated from the particle pellet. The pellet was then resuspended in fresh release buffer until the next time point. Supernatant samples were frozen until the end of the study, at which point ELISA analysis was performed.

Results:

The cytokine-encapsulating NPs were characterized through examination of physical properties, encapsulation metrics, and release kinetics, as described by McHugh et al., Biomaterials, 2015; 59:172-81 and Park et al., Mol Pharm, 2011; 8:143-52). By dynamic light scattering, NPs were found to have a mean±SD hydrodynamic diameter of 245.3+2.2 nm with a low polydispersity index (mean±SD: 0.06±0.01), indicative of a uniform NP population with a relatively tight size distribution. Cytokine encapsulation was measured by ELISA after disrupting the NPs using DMSO. Standard curves were generated using cytokine standards, but all wells were supplemented to contain 5% volume/volume DMSO and the appropriate concentration of empty NPs. Using this method, NPs were found to contain a mean±SD of 7.4±0.4 ng TGF-β and 1.9±0.1 ng IL-2 per mg NP. For TGF-β, the percent encapsulation efficiency was 17.8±1.1; for IL-2 was 9.1±0.4.

Release of TGF-β and IL-2 from the NPs loaded with both cytokines and IL2 from NPs containing only this cytokine exhibited a burst release during the first 24 hours, followed by a slower, more sustained release profile over the course of the tested 14-day period.

### Example 2. Conditions for the Induction of CD4+ and CD8+ Treg Cells In Vitro in Mice with Nanoparticles Containing IL-2 and TGF-β

Materials and Methods: For T cell proliferation, sorted CD3+ T cells (negatively selected with magnetic beads) from 12 week-old BALB/c mouse splenocytes were cultured at 37° C. at a concentration of 2×105 cells/well in 96-well plates (Corning) in complete RPMI medium (100 units/ml penicillin, 100 μg/ml streptomycin, and 10% heat-inactivated fetal calf serum) for 72 hours in the absence (control) or in the presence of plate-bound anti-CD3 antibody (1 μg/ml) and soluble anti-CD28 antibody (1 μg/ml) (BD Biosciences). Treg cells were defined as cells that expressed the transcription factor, Foxp3.

Statistical analyses were performed using GraphPad Prism software version 5.0. Parametric testing was done using the unpaired t-test; nonparametric testing was used when data were not normally distributed. P values less than 0.05 were considered significant.

### Results:

To induce CD4+ and CD8+ Treg cells simultaneously, PLGA NPs encapsulating IL-2 and TGF-β were used in amounts that had been used in McHugh et al., Biomaterials, 2015; 59:172-81. Scalar doses of NPs coated with anti-CD2/CD4 antibodies were added in culture to mouse purified CD3+ cells for the delivery to T cells, in a paracrine manner, of IL-2 and TGF-β that induce Treg cells in vitro. Anti-CD3/CD28 antibody stimulation with 50 μg/ml NPs promoted a significant increase in the frequency of both CD4+ and CD8+ Foxp3+ Treg cells. This stimulation was needed for a maximal increase in Tregs.

### Example 3: Establishment of In Vivo Conditions for the Induction of Therapeutic CD4+ and CD8+ Treg Cells In Vivo in Mice with Nanoparticles Containing IL-2 and TGF-β

### Materials and Methods:

Mice: C57131/6, DBA/2, and BALB/c mice (including DO11.10, H2d) were purchased from the Jackson Laboratory. Mice were monitored to measure the frequency of circulating Tregs by flow cytometry. Serum samples were obtained via retroorbital bleeding. Mice were maintained in specific pathogen-free facilities at the University of California, Los Angeles. Experiments were approved by the Institutional Animal Research Committee.

Flow Cytometry: Performed as described above. Peripheral blood mononuclear cells (PBMCs) or splenocytes were isolated according to standard procedures and single-cell suspensions were used for phenotype analyses using combinations of fluorochrome-conjugated antibodies. After Fc blocking, fluorochrome-conjugated anti-mouse antibodies to CD4, CD8, CD25, CD19, CD11b, CD11c, and Gr-1 (all from BD Biosciences) or isotype control antibodies were used for staining prior to acquisition on a FACS Calibur flow cytometer (BD Biosciences) and subsequent analysis using FLOWJO® software (Tree Star). For intracellular staining of FoxP3, cells were first stained for the expression of cell-surface markers before fixation/permeabilization and FoxP3 staining using the eBioscience FoxP3 Staining Kit, according to the manufacturer's instructions.

PBMSs from 8-10 week old BDF1 mice were gated as B cells (CD19+) granulocytes (Grp, monocytes (CD11b+), dendritic cells (CD11c+), and CD3+ T cells (further divided as CD8+ and CD4+ cells).

In vitro T cell responses to antigenic stimulation was performed in the presence of ovalbumin 323-339 peptide (OVA323-339, ThermoFisher Scientific). Splenocytes form DO11.10 mice were cultured with 10 μg/ml OVA323-339 in the presence or absence of NPs encapsulating IL-2 and TGF-β (either coated or not coated with anti-CD2/CD4 antibodies). 3H-thymidine was added during the last 16 hours before cells harvesting on a Tomtec Harvester 96. Stimulation index was calculated as mean counts per minute (cpm) of antigen-stimulated wells/mean cpm of wells with medium only. These studies were conducted to learn if tolerogenic NPs altered the T cell response to conventional antigens.

Statistical analyses were performed as described previously.

### Results:

Treatment with anti-CD2/CD4-coated NPs was compared with treatment with NPs coated with only anti-CD2 antibody only or anti-CD4 antibody only, keeping constant the total amount of NPs (all encapsulating IL-2 and TGFβ). After a loading dose, 1.5 mg NPs were injected every 3 days or 6 days for the first 12 days. One week later, both groups of mice received another 1.5 mg NPs. Analysis of Treg cells among circulating PBMCs on day 21 revealed that only those animals that had received NPs every three days had significant increases in Treg cells.

Anti-CD4 antibody-coated NPs expanded CD4+CD25+FoxP3+ cells but NP coating with anti-CD2/CD4 enhanced this effect. Importantly, the coating antibodies needed to be attached to the same NPs (co-coated), since coating of anti-CD2 and anti-CD4 antibodies independently on NPs was not effective in expanding Treg cells.

Anti-CD2 antibody-coated NPs also enhanced FoxP3 expression in CD4+ cells, but unlike anti-CD2/CD4-coated NPs, could not increase CD25 expression significantly in this experiment. Anti-CD2 antibody-coated NPs, however, significantly expanded CD8+Foxp3+ cells, and the percentage of CD8+Foxp3+ cells induced by anti-CD2 antibody-coated NPs was higher than that from anti-CD2/CD4-coated NPs. This can be due to lower per-NP coating of anti-CD2 antibody in the co-coated system and increased competitive binding to CD4+ T cells). This treatment for the expansion of Treg cells did not affect overall T cell responsiveness to antigenic stimulation, indicating that the binding of NPs to CD2 or CD4 co-receptors did not impede activation through the T cell receptor.

### Example 4: In Vivo Studies in BDF1 Mice with Lupus with Nanoparticles Containing IL-2 and TGF-β

### Materials and Methods

Flow cytometry and statistical analyses were performed as described previously.

Mice: Female 057131/6 mice and male DBA/2 mice were bred for the generation of (C57Bl/6×DBA/2) F1 (BDF1) mice. At the age of eight weeks, BDF1 mice were induced to develop disease by the transfer of parent DBA/2 cells according to Zheng et al., J Immunol, 2004; 172:1531-9. In the recipient mice, the recognition of the host major histocompatibility complex (MHC) antigens leads to lymphoid hyperplasia and elevated production of anti-double-stranded DNA (anti-dsDNA) antibodies followed by immune-complex glomerulonephritis. Following transfer of DBA/2 cells, BDF1 mice were then given an intraperitoneal (IP) injection of vehicle as control or PLGA NPs encapsulating IL-2 and TGF-β and left uncoated (control) or coated with anti-CD2 and anti-CD4 antibodies (BD Biosciences). Mice were monitored bi-weekly to measure the frequency of circulating Treg cells by flow cytometry. Serum samples were obtained via retroorbital bleeding. Proteinuria was measured using ALBUSTIX® strips (Siemens). Mice were maintained in specific pathogen-free facilities at the University of California, Los Angeles. Experiments were approved by the Institutional Animal Research Committee.

ELISA of Anti-double-strand DNA Antibody: ELISA measurement of anti-double-strand DNA (anti-dsDNA) antibody levels was performed using kits from Alpha Diagnostics International, according to the manufacturer's instructions. Optical density (O.D.) was measured at 450 nm.

Histology: Kidney sections (4-μm thick) were stained with hematoxylin and eosin (H&E) according to Lourenco et al., Proc Natl Acad Sci USA, 2016; 113:10637-42. For assessment of pathologic changes by glomerular activity score and tubulointerstitial activity score, sections were scored in a blinded manner, using a scale of 0-3, where 0=no lesions, 1=lesions in <30% of glomeruli, 2=lesions in 30-60% of glomeruli, and 3=lesions in >60% of glomeruli. The glomerular activity score includes glomerular proliferation, karyorrhexis, fibrinoid necrosis, inflammatory cells, cellular crescents, and hyaline deposits. The tubulointerstitial activity score includes interstitial inflammation, tubular cell necrosis and/or flattening, and epithelial cells or macrophages in the tubular lumen. The raw scores were averaged to obtain a mean score for each feature, and the mean scores were summed to obtain an average score from which a composite kidney biopsy score was obtained (Ferrera et al., Arthritis Rheum, 2007; 56:1945-53). For indirect immunofluorescence studies, sections were fixed in cold acetone for 5 minutes, washed, and blocked with 2% bovine serum albumin (BSA) for 1 hour before staining with rabbit anti-mouse IgG (Fisher Scientific).

Results: When the treatment protocol was followed in BDF1 mice with lupus, treatment with anti-CD2/CD4 antibody-coated NPs encapsulating IL-2 and TGF-β resulted in increased numbers of circulating CD4+ and CD8+ Treg cells. Protection against lupus disease manifestations was observed when a total amount of 7.5 mg of NPs was used.

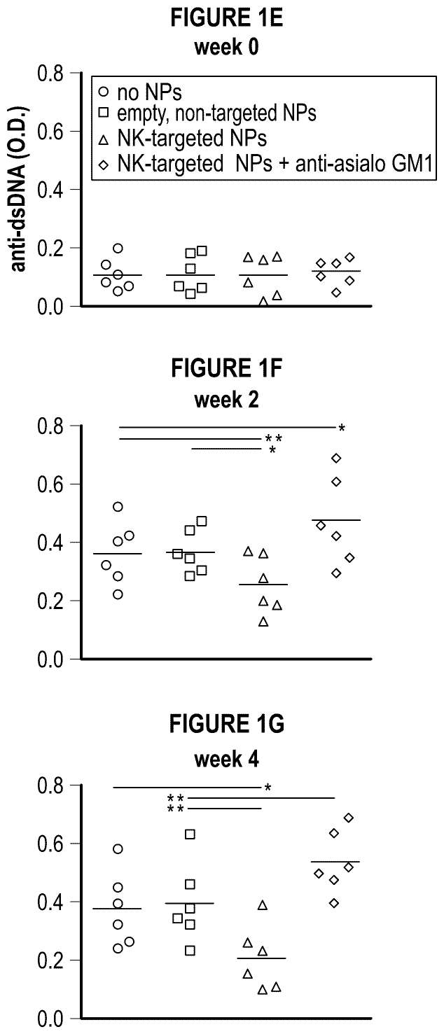

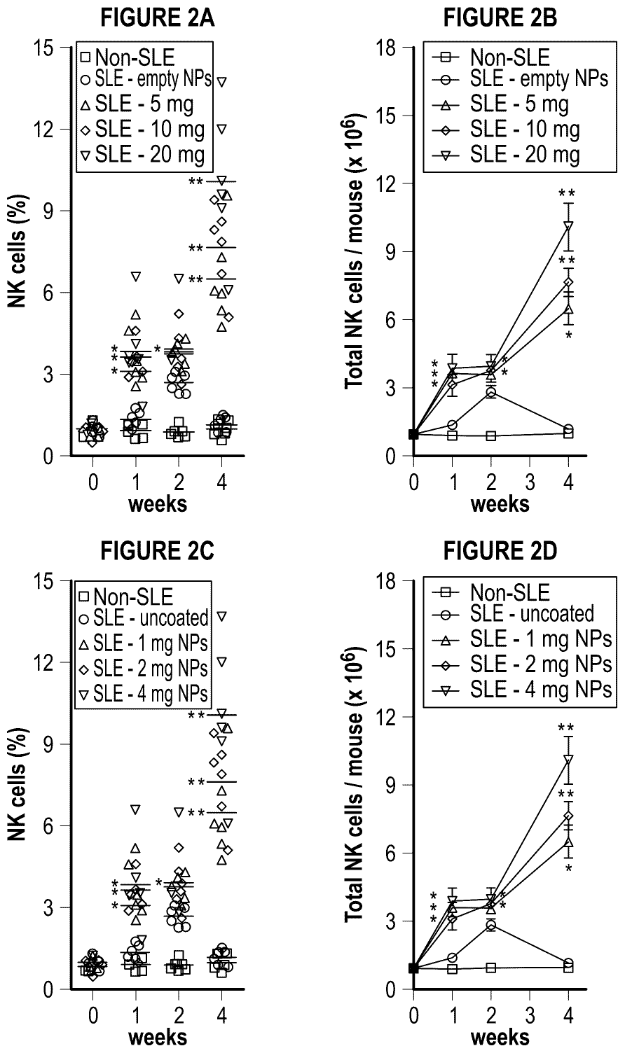

In BDF1 mice, disease onset after transfer of DBA/2 cells is rapid, with anti-DNA autoantibodies appearing by two weeks and proteinuria due to immune complex glomerulonephritis by six weeks after transfer (Via et al., Immunol Today, 1988; 9:207-13; Rus et al., J Immunol, 1995; 155:2396-406; Zheng et al., J Immunol, 2004; 172:1531-9). Mice received 7.5 mg of anti-CD2/4 coated NPs over 19 days. The schedule is shown in FIG. 1A). These NPs markedly increased CD4 and CD8 Tregs (FIGS. 1B-1C). This dose schedule was associated with an increase in CD4+ Treg cells of about two-fold and an increase in CD8+ Treg cells of about four-fold, but not with changes in the frequency of other immune cell populations, and with a statistically significant reduction in the production of anti-dsDNA autoantibodies at week 2 and 4 (FIG. 1E (p<0.05) and decreased proteinuria (FIG. 2C) p<0.05.

Treatment of BDF1 mice with 7.5 mg NPs encapsulating IL-2/TGF-β did not cause significant changes in the frequency of multiple populations of circulating immune cells as compared to BDF1 mice receiving unconjugated NPs.

NPs needed to be targeted for expansion of CD4+ and CD8+ Foxp3-expressing Treg cells and for the protection of mice from developing anti-DNA autoantibodies and proteinuria. Non-coated NPs containing IL-2 and TGF-β administered at equivalent doses had none of these effects. The decreased proteinuria in mice treated with T cell-targeted NPs encapsulating IL-2 and TGF-β was reflected by histopathological kidney changes that indicated preserved glomeruli and reduced IgG precipitation. Conversely, control mice (including those treated with untargeted NPs) displayed glomerular hypercellularity and proliferative changes characteristic of lupus nephritis and IgG precipitation that associated with worse renal disease scores.

In summary, NPs that can expand both CD4+ and CD8+ Treg cells in vivo sufficiently to suppress lupus manifestations in mice has been developed. The coating with anti-CD2/CD4 antibodies enabled NPs to bind both CD4+ and CD8+ T cells for the expansion of both cell types in vivo, in mice without lupus and in BDF1 mice with lupus, with resulting reduction of anti-dsDNA autoantibody levels and immune-complex glomerulonephritis in the latter.

Several tolerogenic strategies enhance the ability of lupus Treg cells to suppress production of pathogenic autoantibodies, including anti-DNA. These include an induction and expansion of Treg cells or the administration of tolerogenic peptides that induce both CD4+ and CD8+ Tregs (Zheng et al., 2005; La Cavaet et al., J Immunol, 2004; 173:3542-8; Singh et al., J Immunol, 2007; 178:7649-57; Kang et al., J Immunol, 2005; 174:3247-55; Sharabi et al., J Immunol, 2008; 181:3243-51; Scalapino et al., PLoS One, 2009; 24:e6031). The immunotherapeutic potential of CD8+ Tregs in SLE has not been examined thoroughly, although it is known that improved function of CD8+ Tregs in human SLE is associated with disease remission (Suzuki et al., J Immunol, 2012; 189:2118-30; Zhang et al., J Immunol, 2009; 183:6346-58). IL-2 and TGF-β can induce CD8+ cells to become Tregs (Hirokawa et al., J Exp Med, 1994; 180:1937), with a protective activity in humanized mice (Horwitz et al., Clin Immunol, 2013; 149:450-63). When both CD4+ and CD8+ Tregs induced ex vivo were used with IL-2 and TGF-β to suppress lupus-like disease in BDF1 mice, the therapeutic effects were much stronger than when the mice were treated with CD4+ Tregs alone, demonstrating an important role of CD8+ Tregs in suppressing lupus autoimmunity (Zheng et al. J Immunol, 2004; 172:1531-9).

Mechanistically, the observed interaction of anti-CD2 and anti-CD4 Ab demonstrates two non-mutually-exclusive possibilities: 1) antibody administration to target cells with nanoscale reagents affords multivalency (i.e., multiple copies of antibodies binding the targets would increase avidity, and thus pharmacological effects); and 2) targeted proximal release of IL-2 and TGF-β promotes local expansion of Tregs. In this context, the encapsulant released from NPs is most effective within nanoscale distances from the target cell.

The “flattening” of the cell interface was previously mathematically modeled as it interacts with the particle, showing a significantly enhanced magnitude of cytokine accumulation at the cell-particle interface (Labowsky et al., Nanomedicine, 2015; 11:1019-28; Labowsky et al., Chem Eng Sci, 2012; 74:114-123; Steenblock et al., J Biol Chem, 2011; 286:34883-92). This phenomenon of “paracrine effect post-release” suggests that targeting, and therefore ligation, via anti-CD2 and anti-CD4 Ab can bring particles and T cells within nanoscale ligand receptor distances, increasing local concentration of cytokines capable to act on cells with great efficacy (McHugh et al., Biomaterials, 2015; 59:172-81). This phenomenon has been validated in systems for artificial antigen presentation, which have shown that IL-2 encapsulated in NPs has an equivalent T cell stimulatory effect to soluble IL-2 at 1000-fold higher concentration. Additionally, NPs create a local acidic microenvironment that can convert endogenous latent TGF-β to its active form, and this could enhance IL-2 in extending Tregs expansion, even after the TGF-β stores in the NPs are depleted. Taken together, these features demonstrate an advantage in the use of nanoparticulate delivery systems to afford cytokine delivery at local levels in minute doses, mitigating high dose related toxicity while retaining high bioactivity.

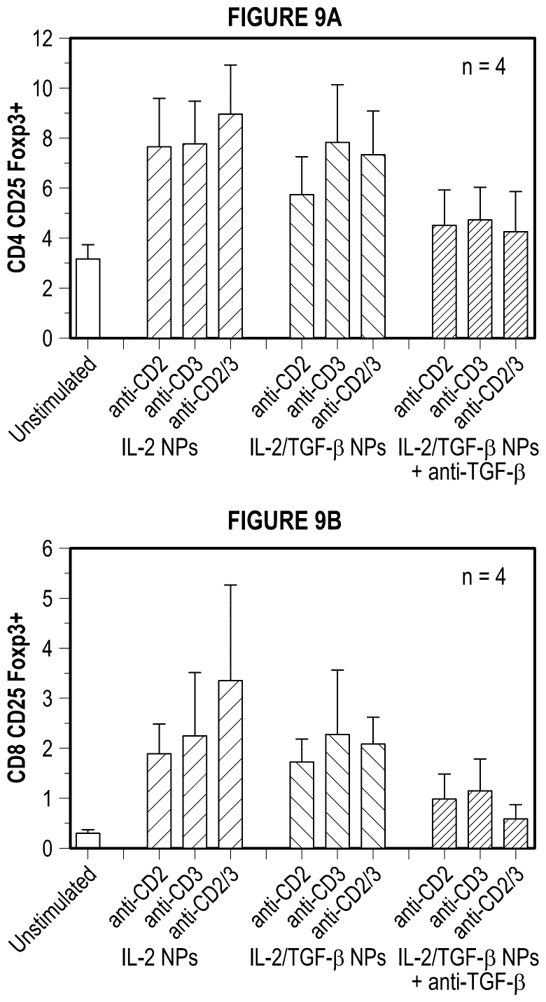

Since CD2 is also expressed by NK cells, the effect of NK cell depletion on the severity of the lupus-like-syndrome was determined. Symbols represent the different groups of mice (n=6 per group); error bars show the mean±SEM. FIG. 1B shows the percentages of peripheral CD4+(FIG. 1B) and CD8+(FIG. 10) Tregs at the indicated time points after treatment. FIGS. 1B and 10 show that depletion of NK cells reduces the expansion of CD4+ and CD8+ Tregs induced by NPs loaded with IL-2 and TGF-β and decorated with anti-CD2/CD4 antibodies *P<0.05 and **P<0.05 in the comparison between empty NPs versus cytokine-loaded NPs; § P<0.04 between mice depleted (anti-asialo GM1, a-asGM1) or not of NK cells. These studies revealed that NK cells support the increase in CD4 and CD8 Tregs and are intimately involved in the protective effects of the tolerogenic NPs. FIG. 1D show proteinuria at the time points indicated for the mice in FIG. 1B-C. Depletion of NK cells not only abolished the protective effects of the NPs, but also significantly exacerbated renal disease (**P<0.005 in the comparison between mice treated with cytokine-loaded NPs depleted (aaGM1) or not of NK cells). These results demonstrate that NK cells modulate the tolerogenic activity of the NPs in BDF1 mice.

In summary, the therapeutic effects of anti-CD2/4 coated NPs was dependent upon NK cells. NK cell depletion not only inhibited the increase in Tregs and their protective effects, but also increased the severity of the disease. These results prompted further studies on the role of protective NK cells. NK have immuno-modulatory properties in addition to their cytotoxic properties). NK cells express high levels of CD2 molecules on their cell surface. Anti-CD2 can stimulate NK cells to produce TGF-β (Ohtsutka and Horwitz, J Immunol 160:2539-45, 1998) and inhibit B-cell production of antibodies via TGF-β. aAPCs coated with anti-CD2 could have much more persistent effects that soluble anti-CD2. These aAPCs, then, could induce and sustain potent suppressive regulatory NK cells.

### Example 5: Role of a TGF-β Dependent NK Cell Induced by Targeted Tolerogenic Artificial Antigen-Presenting Nanoparticles (aAPCs) in the Protecting BDF1 Mice from Lupus Nephritis

### Materials and Methods:

The BDF1 mice are the same as in the previous examples.

The NPs had been either left uncoated (control) or in continuation of the experiments indicated in example 4, they were decorated with biotinylated anti-CD2 antibody and biotinylated anti-CD4 antibody (clone GK1.5, Thermo Fisher Scientific). Initially they were and loaded with IL-2 and TGF-β. However, since anti-CD2 can induce NK cells to produce TGF-β, the later experiments were with NPs coated with only anti-CD2 and loaded with only IL-2.

Lupus-like disease was induced at 8 weeks of age, according to standard protocols, by transferring 1×10⁸ DBA/2 splenocytes into BDF1 mice. After the transfer of the DBA/2 splenocytes, individual BDF1 mice were given intraperitoneal (i.p.) injections of vehicle (as control) or 1 mg PLGA NPs loaded with IL-2/TGF-β or IL-2. As before, the protocol of NPs administration was the following: day 0, day 3, day 6, day 9, day 12 and day 19.

In a series of experiments, mice received i.p. 100 μl of NK-depleting anti-asialo GM1 or control rabbit sera (Wako Chemicals, Richmond, VA) at 4-days intervals. Efficacy of NK depletion of greater than 90% was assessed by flow cytometry using FITC-labeled anti-NK1.1 antibody (clone PK136, Thermo Fisher Scientific). Mice were monitored at weekly intervals using blood obtained via retroorbital bleeding for analyses that included flow cytometry on circulating immune cells and ELISA measurements of serum anti-dsDNA antibodies (Alpha Diagnostic Intl., San Antonio, TX) and creatinine (Abcam, Cambridge, MA). Proteinuria was measured using Albustix strips (Siemens Diagnostics, Irvington, NJ). In a series of experiments, individual mice received i.p. every other day from day 0, for two weeks, 100 μg anti-TGF-μg antibody (clone 1D11.16.8—a neutralizing antibody to all three isoforms of TGF-β that has a circulating half-life of 15.2 hours or the same amount of isotype control antibody (clone P3.6.2.8.1) (both from Novus Biologicals, Centennial, CO). All experiments with mice were approved by the institutional Animal Research Committee.

Flow Cytometry: Peripheral blood mononuclear cells (PBMCs) or splenocytes were isolated according to standard procedures, and single-cell suspensions were used for phenotypic analyses following red blood cell lysis. After Fc blocking, anti-mouse antibodies to NK1.1 (FITC-labeled) or H-2Kb/H-2db (PE-labeled) (clone 28-8-6, Biolegend, San Diego, CA) or isotype control antibodies were used for staining. After acquisition on a FACSCalibur™ flow cytometer (BD Biosciences, San Jose, CA), data analysis was done using FlowJo™ software (BD, Franklin Lakes, NJ).

siRNA Transfection and Real-time PCR: The protocol of siRNA transfection. Briefly, untouched NK cells isolated using the NK Cell Isolation kit on an autoMACS (Miltenyi Biotec, Auburn, CA) were plated on 12-well plates in complete medium containing 10% fetal bovine serum 24 hours before transfection with the Silencer Select siRNA for mouse Tgfb1 (Thermo Fisher Scientific) using the Silencer siRNA Transfection II Kit that also included GAPDH siRNA as positive control and a negative control siRNA with no significant sequence similarity to mouse, rat, or human gene sequences (Silencer siRNA Transfection II Kit). siPORT amine transfection agent was diluted in OptiMEM™ medium (Thermo Fisher Scientific) and used alone as additional control or mixed with 10 nM siRNAs (Tgfb1 or controls) before incubation for 30 min. at room temperature. Sorted NK cells were transfected with the siRNA complexes before transfer into BDF1 mice. To control efficiency of siRNA transfection before the adoptive transfer, a small aliquot was lysed with TRIzol™ reagent (Thermo Fisher Scientific) for total RNA isolation. 100 ng RNA were used with one-step RT-PCR reagents from Thermo Fisher Scientific using primers and probe combinations as described. For relative quantitation, a standard curve was constructed for each primer and probe set, using total RNA. GAPDH was used as an endogenous control in each experimental set. All samples were run in duplicate.

Statistical Analyses: Statistical analyses were performed using GraphPad Prism software (version 5.0). Parametric testing was done using the Student's t-test; nonparametric testing was used when data were not normally distributed. P values less than 0.05 were considered significant.

### Results

NK Cells Expand in Nanoparticle-Treated BDF1 Lupus Mice and are Host-Derived

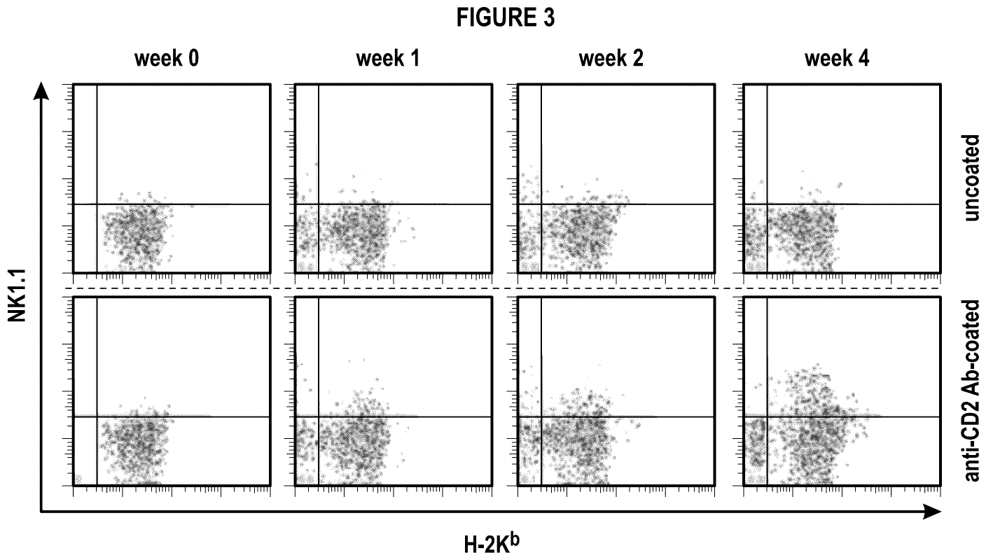

FIGS. 2A-2D document that NK cells show a dose-dependent expansion in BDF1 with lupus-like disease after treatment with CD2-targeted NPs loaded with IL-2 and TGF-β. Controls were uncoated NPs loaded with IL-2 and TGF-β and empty uncoated NPs. FIG. 3A shows the percentages of circulating NK cells among the PBMCs in individual untreated BDF1 mice (“Non-SLE”, squares) or lupus BDF1 mice treated with different doses of NPs encapsulating IL-2 and TGF-β (circle, empty NPs; triangle 5 mg; diamond 10 mg; inverted triangle 20 mg). FIGS. 2B and 2D shows the total numbers of NK cells with mean+SE in mice with the same treatments. P in the comparison with SLE BDF1 mice treated with empty NPs=*<0.005,**0.0005.

FIG. 3 shows that NK cells that expand in BDF1 lupus mice after treatment with NPs are host-derived (H-2Kb+). To understand whether the expanded NK cell population derived from the host (BDF1 mice) or from the donor (DBA/2 mice), flow cytometry was used to assess the surface expression of H-2 molecules on the expanded NK cells. The parental haplotypes of the recipient BDF1 lupus mice are H-2b (C57BL/6) and H-2d (DBA/2), so the transferred H-2d splenocytes from DBA/2 mice do not stain with anti-H-2b antibodies. Therefore, H-2b NK cells must only be of host origin. Flow cytometry analyses showed that NP administration increased the number of H-2b (host) NK cells at two weeks, expanding further at four weeks (FIG. 2). There was neither an increase in circulating H-2b cells in BDF1 mice that did not receive NPs nor an increase in H-2d donor NK cells. Thus, the expansion of NK cells in NP-treated BDF1 lupus mice was the result of an increase in the (relative and absolute) numbers of host-derived NK cells.

NP-Mediated Expansion of NK Cells Associates with the Suppression of Anti-dsDNA Antibodies and Reduced Lupus Disease Manifestations in BDF1 Mice.

Because of the central role of autoantibodies in lupus pathogenesis and the finding that NK cells can suppress B-cell production of antibodies in vitro and in vivo, the possible influence of NK cells on autoantibody levels in BDF1 lupus mice was investigated. Moreover, since anti-CD2 antibodies induce NK cells to produce TGF-β, the possibility that the production of this cytokine by NK cells could substitute for that encapsulated in the NPs was assessed. In experiments with NPs that contained only IL-2, NK cells markedly influenced the serum levels of autoantibodies in BDF1 lupus mice that received NPs (FIG. 2A-2C).

FIG. 1E-1G shows depletion of NK cells in BDF1 lupus mice abolished the protective effects of CD2 (NK)-targeted NPs loaded with IL-2 and was associated with increased levels of serum anti-dsDNA autoantibodies. FIGS. 1E-1G shows that treatment of BDF1 mice with CD2 (NK)-targeted NPs loaded with IL-2 associates with suppression of anti-DNA autoantibodies. Depletion of NK cells in these mice by administering anti-asialo GM1 not only abolished the protective effect of the NPs, but also associates with increased levels of serum anti-dsDNA autoantibodies. Symbols: Circle, no NPS but PBMCs; square empty, non-targeted NPs, triangle NK-targeted NPS, and solid triangle NK-targeted NPS and anti-asialo GM1 plotted against levels of anti-dsDNA (absorbance) Monitoring of individual mice and group means are reported at week 0 (FIG. 1E), 2 weeks (FIG. 1F), and 4 weeks (FIG. 1G) post-induction of SLE (time 0). *P<0.05, **P<0.01.

FIG. 4A demonstrates protection from lupus nephritis of BDF1 mice treated with CD2 (NK)-targeted NPs depends on NK cells. FIG. 4A shows NK cell depletion accelerates proteinuria in BDF1 lupus mice. NK cells were depleted by administering 100 μl anti-asialo GM1 every 4 days for 2 weeks from day 0 (induction of SLE). Mice (n=6 per group) were monitored for 8 weeks post-induction of SLE. Data show the mean+SE; *P<0.01 at 4 and 6 weeks in the comparison between BDF1 mice receiving NK cell-targeted NPs with or without NK-depleting anti-asialo GM1 and at 4 weeks between mice receiving empty, non-targeted NPs versus mice depleted of NK cells.

The Identification of TGF-β-Dependent NK Cells that have Beneficial Effects on the Renal Manifestations in BDF1 Lupus Mice

NK cells can be divided into two major groups. Most are killer cells, but there is a subset that primarily produces cytokines. Most produce large amounts of interferon γ (IFN-γ), but some have been described that produce IL-10 or TGF-β. To learn whether TGF-β contributes to the suppression of the autoimmune response in BDF1 lupus mice, the effects of TGF-β inhibition on lupus nephritis in BDF1 mice was tested. The readout in these experiments was the measurement of serum creatinine levels. Increased serum creatinine is an early indicator of kidney injury and reflects a progression to renal insufficiency and to end-stage renal disease in lupus nephritis. The comparison between BDF1 lupus mice that received NK-targeted NPs together with anti-TGF-β antibody versus mice that received an irrelevant control antibody indicated that the inhibition of TGF-β associated with a significant increase in serum creatinine levels (FIG. 4B). The contributing role of NK cells was confirmed by the finding of elevated serum creatinine in BDF1 lupus mice that had been depleted of NK cells with anti-asialo GM1 (FIG. 4B). The finding that the combination of anti-asialo GM1 and anti-TGF-β antibodies did not influence serum creatinine levels indicated a common mechanism (FIG. 4B).

To test the possibility, NK cells were the source of TGF-β that protected BDF1 mice from renal disease, mice were treated with NK-targeted NP and then sorted for the adoptive transfer to mice who were developing lupus nephritis. The ability of NK cells to produce TGF-β in some was abolished by siRNA technology. Controls received transcription of scrambled siRNA. The adoptive transfer of 2.5×10⁶ TGF-β sufficient NK cells into BDF1 lupus mice protected the mice from renal disease, with no increase of serum creatinine levels (FIG. 4C). No protection was present in BDF1 mice receiving an identical number of GF-β deficient (TGF-β siRNA) NK cells (FIG. 4C). Together, these results demonstrate that the disease-protective effects of NK cells in BDF1 mice are TGF-β-dependent.

### Example 6: Establishment of Conditions for the Induction of Human CD4+ and CD8+ Tregs with aAPCs

### Materials and Methods

Preparation of PLGA Nanoparticles: Poly lactic-co-glycolic acid (PLGA) nanoparticles (NPs) were prepared as described above. After preparation, the NPs were characterized through examination of physical properties, encapsulation metrics, and release kinetics. By dynamic light scattering, NPs were found to have a mean±SD hydrodynamic diameter of 245±2 nm with a low polydispersity index indicative of a uniform NP population with a relatively tight size distribution. Cytokine encapsulation was measured by ELISA after NPs were disrupted using DMSO, and standard curves were generated using cytokine standards with all wells supplemented to contain 5% volume/volume DMSO and the appropriate concentration of empty NPs. NPs contained a mean±SD of 7.4±0.4 ng TGF-β and 1.9±0.1 ng IL-2 per mg of NP. For cell targeting, NPs diluted in PBS were incubated 10 minutes prior to use with the relevant biotinylated targeting antibody (anti-CD4, -CD8 or CD3) at a concentration ratio of 2 μg antibody/mg NP.

Isolation of Human Peripheral blood mononuclear cells (PBMCs): Human PBMCs were prepared from heparinized venous blood of healthy adult volunteers by Ficoll-Hypaque density gradient centrifugation and used fresh for transfer experiments or cultured for 5 days in U-bottom well plates at a concentration of 0.5×10⁶/well in complete AIM V™ medium (Thermo Fisher Scientific, Waltham, MA). All protocols that involved human blood donors were approved by the IRB at the University of California Los Angeles. In some experiments, PBMCs were cultured with anti-human CD3/CD28 DYNABEADS® (Thermo Fisher Scientific) or with IL-2 (100 U/ml) and TGF-β (5 ng/ml) or anti-TGF-8 (1D11) (all from R&D Systems, Minneapolis, MN). In vitro suppression assays were performed according to standard protocols. CD4+CD25− T cells isolated by negative selection to a purity of >95% using the Miltenyi Biotec CD4+CD25+CD127dim/Regulatory T Cell Isolation kit II served as responder cells in cocultures for 3 days with Tregs (positive fraction) isolated with the same kit, following the manufacturer's instructions. Culture supernatants were analyzed for IFN-γ content by ELISA (R&D Systems). Proliferation was evaluated by a liquid scintillation counter following addition of 3H-thymidine (1 μCi/well) 16 hours before analysis.

Flow Cytometry: Human PBMCs or magnetic-bead sorted cells were stained following standard procedures with the following FITC-, PE-, PerCP- or APC-conjugated anti-human antibodies: CD4 (RPA-T4), CD8 (RPA-T8), CD25 (MEM-181), CD127 (eBioRDR5), FoxP3 (PCH101), or isotype controls. All antibodies were from Thermo Fisher Scientific. Data were acquired on a FACSCalibur™ flow cytometer (BD Biosciences, San Jose, CA) and analyzed using FlowJo™ software (BD, Franklin Lakes, NJ).

Mice: The human-to-mouse xenogeneic graft versus host disease (GvHD) model, in which the disease develops in recipient NOD/scid/IL2r common γ chain−/− (NSG) mice following the transfer of human PBMCs. NSG mice were purchased from the Jackson Laboratory (Bar Harbor, ME) and housed under specific pathogen-free conditions in microisolator cages with unrestricted access to autoclaved food and sterile water. 10⁷ fresh human PBMCs were resuspended in 200 μl of PBS in insulin syringes and injected i.v. via the tail vein into individual unconditioned NSG mice of 8-12 weeks of age. The mice also received i.v. (individually) 1.5 mg IL-2/TGF-β-loaded NPs decorated with anti-CD3 (OKT3, Thermo Fisher Scientific), starting on the day of transfer of human PBMCs: day 0, 3, 6, 9, 12. Control mice received empty uncoated NPs or PBS under identical conditions as the above NP-treated mice. The experiments were performed according to the guidelines of the Institutional Animal Committee of the University of California Los Angeles. Animals that developed hunched posture combined with lethargy and/or lack of grooming, reduced mobility or tachypnea, were euthanized and an end-point of survival was recorded at the time of sacrifice. Disease was monitored using a validated scoring system that evaluates each of the five following parameters as 0 if absent or 1 if present: 1) weight loss >10% of initial weight; 2) hunching posture; 3) skin lesions (patchy alopecia); 4) dull fur; 5) diarrhea. Dead mice received a total score of 5 until the end of experiment. Peripheral blood (to separate PBMCs for flow cytometry) and plasma were collected on days 0, 4 and 14 and 50. Plasma concentrations of human IgG were measured by ELISA (Thermo Fisher Scientific). For histologic evaluations, lung, liver and colon were collected on day 50 after the transfer of PBMCs. Tissues were fixed in formalin, paraffin embedded, and sections stained with hematoxylin/eosin.

Statistical Analyses: Statistical analyses were performed using GraphPad Prism software version 5.0. Parametric testing was done using the Student's t-test; nonparametric testing was used when data were not normally distributed. Differences in animal Kaplan-Meier survival curves were analyzed by the log-rank test. P values less than 0.05 were considered significant.

Results: Paracrine Delivery of Cytokines to Human T Cells by aAPC NPs Leads to the Induction and Expansion of Functional Tregs

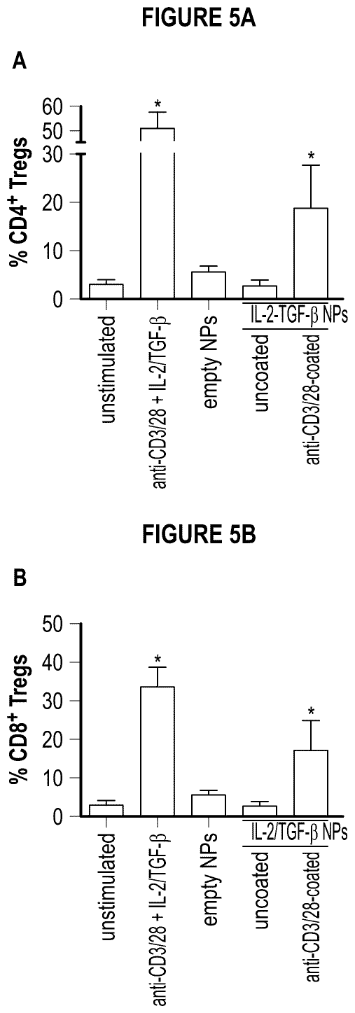

It was investigated whether the NPs could induce a tolerogenic T-cell program without the engagement of the T-cell co-receptors CD4 or CD8, i.e. by acting as tolerogenic aAPCs delivering tolerogenic cytokines to human T cells in the presence of TCR stimulation. The NPs loaded with tolerogenic cytokines and coated with anti-CD3/28 antibodies efficiently expanded CD4+ (FIG. 5A) and CD8+ human Tregs (FIG. 5B), in vitro indicating that NPs can operate as acellular aAPCs that can induce the differentiation of human T cells into Tregs.

Having found that the delivery of IL-2 and TGF-β to T cells by the NPs allowed human T cell differentiation into Tregs, the relative contribution of TGF-β to the process was assessed by evaluating its role in the NP-mediated expansion of the Tregs. Parallel cultures including anti-TGF-β Ab or irrelevant control antibody compared the Treg numbers induced and expanded from human PBMCs incubated with IL-2/TGF-β-loaded NPs and decorated with anti-CD3/28.

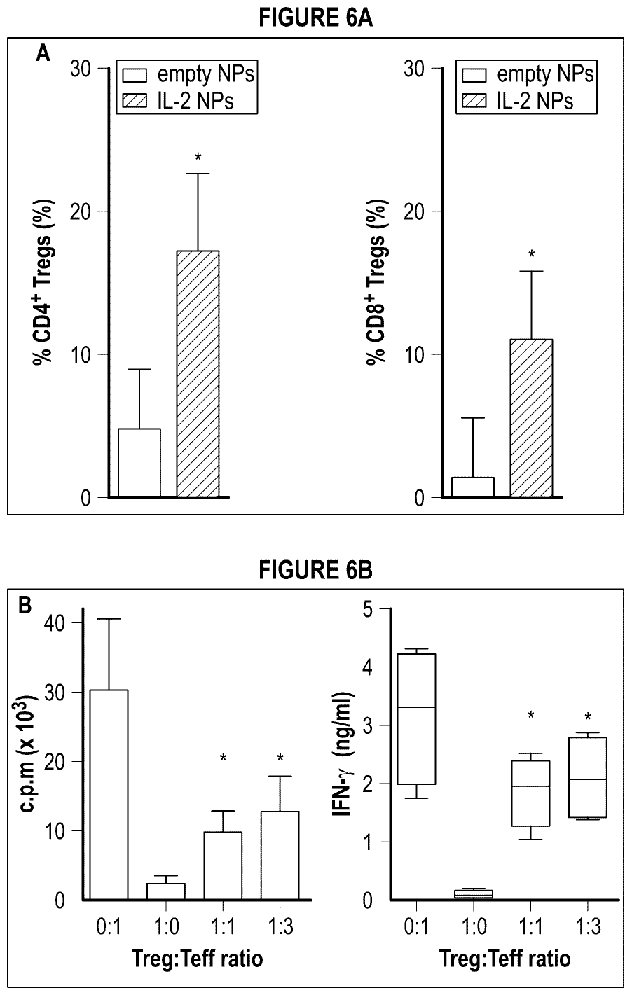

FIG. 6A shows additional evidence that TGF-β did not have to be encapsulated in the nanoparticles. NPs loaded with only IL-2 induced CD4+ and CD8+ Foxp3+ Tregs. The aAPC NP-induced Tregs were functional, as indicated by their ability to suppress in vitro the proliferation and production of proinflammatory cytokines from T effector cells (FIG. 6B).

### Example 7: Induction of Tregs In Vivo by aAPC NPs that Protect Humanized NSG Mice from Human Anti-Mouse Graft Versus Host Disease

The suppressive activity of the Tregs in vitro might not correlate with a suppressive activity in vivo. Taking advantage of the known protective effects of the Tregs in allograft rejection, immunotherapeutic potential of the aAPC NPs was tested in a mouse model of human-anti-mouse GvHD.

### Materials and Methods

Immunodeficient NOD SCID (NSG) mice were also used in these experiments. NSG mice were divided into two groups of 6 mice each. Both groups received i.v. 10⁷ human PBMCs. The human T cells will cause a lethal human anti-mouse graft versus host disease. One group of mice also received aAPC NPs decorated with anti-CD3 containing IL-2 and TGF-β (solid circles) and the other group received empty NPs (open circles). These were given starting on the day of transfer of human PBMCs on days 0, 3, 6, 9, 12.

### Results:

FIGS. 7A-7C show that mice that received T-cell targeted NPs encapsulated with IL-2 had an in vivo expansion of both CD4+ (FIG. 7A) and CD8+ Tregs (FIG. 7B) and that unlike the control mice that received only empty NPs, human IgG did not increase (FIG. 7C).

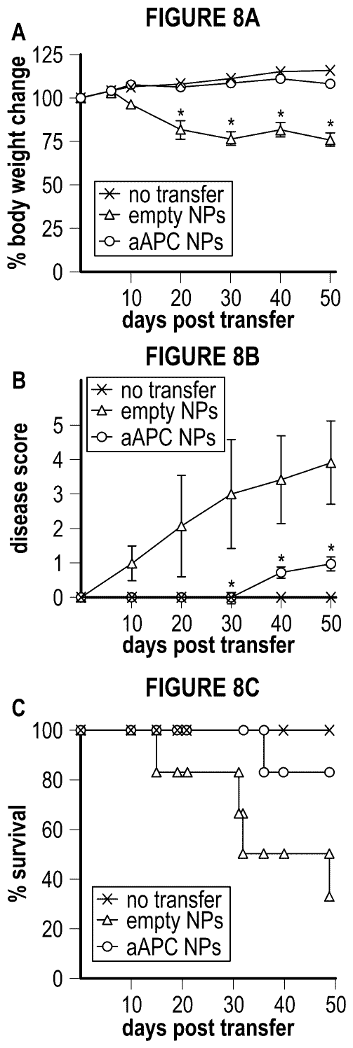

FIGS. 8A-8C show the efficacy of aAPC-NP treated mice. The aAPC NP-protected mice did not lose weight after transfer of the human PBMCs (FIG. 8A), decreased disease score (FIG. 8B), had an extended survival (FIG. 8C) as compared to the mice that had not received NPs or that had received empty NPs. Mice that received the aAPCs did not develop the skin manifestations of GVHD (FIG. 8D). Finally, the histopathology of the lung, liver and colon of NSG mice receiving aAPC NPs showed significant protection as compared to the control mice (FIG. 8E).

### Example 8: Induction of Tregs In Vivo by aAPC NPs that Prevent Rejection of a Foreign Solid Organ Transplant

### Materials and Methods

Preparation of PLGA Nanoparticles: Same as example 6
Isolation of Human Peripheral blood mononuclear cells (PBMCs): Same as example 6
Flow Cytometry: Same as example 6

Results: A 49-year-old male with chronic renal failure received the kidney from a haploidentical sibling. A mixed lymphocyte response conducted before the transplant revealed that the recipient's CD4+ T cells proliferated in the presence of donor non-T cells. Three days before the transplant he received a dose of anti-CD2 coated PLGA NPs loaded with IL-2. On the day of the transplant he received another dose, and this dose was repeated every three days for three weeks. On the day of the transplant he received 50 mg solumedrol to minimize the inflammatory response associated with the procedure. The steroids were then tapered during the next few days and stopped at the end of the week. Following the administration of the NPs there was a significant rise in CD4+ and CD8+ Foxp3+ Tregs and NK cell numbers in the peripheral blood. The grafted kidney was fully functional following the transplant and only a subsequent minimal rise in serum creatinine which returned to normal. There was no need for the introduction of costimulatory molecule blockade and sirolimus to treat a rejection episode. When the recipient's CD4+ T cells were stimulated in vitro post-transplant with donor non-T cells, there was no proliferative response. However, when the CD4+CD25+ Tregs were depleted from the responder cells, the proliferative response returned. Weekly to bi-weekly subcutaneous NPs were required to provide the continuous stimulation and cytokines needed to prevent a rise in serum creatinine.

### Example 9. Nanoparticle Tolerogenic Antigen-Presenting Cells (aAPCa) Containing IL-2 Only that Induce the TGF-β in the Local Environment Needed for the Generation of Human CD4 and CD8 Tregs

### Materials and Methods

Human peripheral blood mononuclear cells (0.5×10⁶/well were cultured in U-bottom 96-well plates. The cells were stimulated with NPs coated with anti-CD2, anti-CD3 or anti-CD2/3 NPs containing IL-2 or IL-2 and TGF-β (50 ug/ml. Some wells contained anti-TGF-β LAP 10 ug/ml. Controls were unstimulated PBMC. The cells were cultured for 5 days and the percentage of CD4 and CD8 cells staining for CD25 and Foxp3 was determined.

Statistical analyses were performed using GraphPad Prism software version 5.0.

Results: NPs coated with either anti-CD2, anti-CD3 or a combination of both increased CD25 and Foxp3 expressed by CD4 and CD8+ cells. NPs containing IL-2 only increased Foxp3 more than NPs containing IL-2 and TGF-β. However the addition of anti-TGF-β abolished this effect. The graph shows the mean of 4 separate experiments. The increases in Foxp3 resulting from the addition of NPs were significant p<0.05 as was the effect of anti-TGF-β p<0.05. ((FIGS. 9A, 9B). Anti-CD3 (Fab′)2 decorated NPs encapsulated with only IL-2 were also capable of inducing Tregs. FIGS. 10A, 10B show that these aAPCs also markedly increased CD4 and CD8 Tregs (*p<0.01). Thus, both anti-CD2 and anti-CD3 decorated NPs loaded with only IL-2 can induce the TGF-β in the local environment needed for the generation of CD4 and CD8 Foxp3+ Tregs.

These examples document that PLGA NPs can be used as acellular aAPCs for the in vitro and in vivo expansion of functional human Tregs as well as mouse Tregs. When the APCs engage the TCR through the MHC/antigen complex and provide costimulatory signals to T lymphocytes, cell differentiation and functional activation ensue. The replication of this process by aAPCs, used as synthetic platforms, can recapitulate the natural interaction between APCs and T cells, allowing the delivery of signals to T cells and the initiation of adaptive immune responses that can include a paracrine delivery of IL-2 to T cells (as in the aAPCs). Employing aAPCs that encapsulate a payload for the promotion of a tolerogenic immune response has significant immunotherapeutic potential effect. The fact that PLGA is biocompatible and has shown a favorable safety profile in clinical settings further envisions the possibility of a rapid translational potential to the clinic of this approach.

While the expansion of human Tregs with aAPC NPs had the advantage of limiting the deleterious effects associated with the in vivo induction of Tregs through systemic treatments with cytokines that carry non-targeted actions, these NPs did not include components of antigen specificity. The induction of polyclonal and non-antigen-specific Tregs might be advantageous in conditions such as SLE, where the chronic systemic autoimmune response has to target multiple self-antigens and polyclonal Tregs suppress the disease, dissimilarly from the paramagnetic iron-dextran NPs expressing peptide/MHC together with anti-CD28 antibodies.

This strategy can have multiple applications for the therapeutic use of Treg-based approaches. In general, the small numbers of Tregs that circulate in the peripheral blood requires an expansion of Tregs ex vivo before infusion in vivo in sufficient numbers. This associates with significant costs and specific technical requirements. Additionally, repeated treatments for the patient are often required, since ex vivo-expanded Tregs can become instable over time. Additionally, chronic inflammation in autoimmune patients promotes the reversal of the phenotype of the transferred Tregs into T effector cells, and Treg potency may decrease over time. Instead, aAPC NPs can provide a sustained Treg activity with prolonged efficacy, as shown in humanized mice, representing a new immunotherapeutic modality for the expansion in vivo of human Tregs that suppress proinflammatory responses in autoimmune settings.

In summary: All immune-mediated disorders are characterized by aberrant immune cells that cause tissue injury. These cells have escaped the control of the regulatory cells that should suppress them. The novel acellular antigen-presenting cell nanoparticles described function as acellular antigen-presenting cells (aAPCs) that target in vivo T cells, or T cells and NK cells. The aAPCs provide them with the stimulation and cytokines that induce them to become functional regulatory cells. The continued use of these aAPCs will expand these regulatory cells and enable them to reach numbers that regain control over the aberrant immune cells. This strategy “resets” the immune system to terminate immune disorder. Moreover, this novel approach avoids the use present immunosuppressive and biological agents which carry severe adverse side effects.

### Example 10: Nanoparticles Containing IL-2 and TGF-β to Prevent or Treat Rejection of Foreign Organ Grafts

Two weeks before the transplant of a MHC mismatched foreign organ the recipient will receive every three days doses of nanoparticles decorated with anti-CD2 that contain IL-2 and TGF-β and subcutaneous injections of MHC peptides that match the donor. These procedures will generate alloantigen specific Tregs that can be demonstrated in a mixed lymphocyte reaction between the donor and recipient. The recipient's T cells will not proliferate in the presence of donor APCs. The presence of Tregs is demonstrated by depleting CD25+ cells in donor PBMCs. This removal will permit the recipient T cells to now respond to donor APCs. With this evidence of T cell non-responsiveness to donor alloantigens, the organ transplant should survive in the recipient with minimal immunosuppression. After the transplant, repeated administration of subcutaneous MHC peptides will boost the number alloantigen-specific Tregs that sustain tolerance (Zheng S G et al. International Immunol. 18:279-89) 2006. Concurrent use of immunosuppressive drugs should not be necessary. Weekly determination of serum IL-2 receptors after the organ transplant will show an increase if transplant rejection is occurring to indicate the need for further nanoparticle and MHC peptide therapy. (Rasool R. Int. J. Organ Transplantation Med 6:8-13, 2015).

Unless defined otherwise, all technical and scientific terms used herein have the same meanings as commonly understood by one of skill in the art to which the disclosed invention belongs. Those skilled in the art will recognize or be able to ascertain using no more than routine experimentation, many equivalents to the specific embodiments of the invention described herein. Such equivalents are intended to be encompassed by the following claims.

1. A method of treating or preventing an immune-mediated disorder in a patient comprising administering to the patient a tolerogenic artificial Antigen Presenting Cell (aAPC) composition comprising: (i) at least one synthetic polymeric nanoparticle, (ii) at least one targeting agent, and (iii) at least one stimulating agent.

2. (canceled)

3. The method of claim 1, wherein the at least one targeting agent targets T cells.

4. The method of claim 1, wherein the at least one targeting agent targets NK cells.

5. The method of claim 1, wherein the at least one targeting agent targets T cells and NK cells.

6. The method of claim 1, wherein the at least one targeting agent targets NKT cells.

7. The method of claim 1, wherein the at least one targeting agent targets CD3.

8. The method of claim 1, wherein the at least one targeting agent targets CD2.

9. The method of claim 1, wherein the at least one targeting agent targets CD3 and CD2.

10. The method of claim 1, wherein the at least one targeting agent induces cells in the patient to produce TGF-β in the local environment.

11. The method of claim 1, wherein the at least one targeting agent is an antibody.

12. The method of claim 1, wherein the at least one targeting agent is at least one member selected from the group consisting of: an anti-CD2 antibody, an anti-CD3 antibody, and an anti-CD3 antibody with an inactivated or absent Fc fragment.

13. The method of claim 1, wherein the at least one targeting agent is an aptamer.

14. The method of claim 13, wherein the aptamer binds TCR-CD3.

15. The method of claim 1, wherein the at least one stimulating agent comprises a cytokine.

16. The method of claim 1, wherein the at least one stimulating agent comprises IL-2.

17. The method of claim 1, wherein the at least one stimulating agent is encapsulated.

18. The method of claim 1, wherein the method induces lymphocytes in the patient to become multiple populations of functional regulatory cells.

19. The method of claim 1, wherein both CD4 and CD8 cells in the patient are induced to become Foxp3+ T regulatory cells.

20. The method of claim 1, wherein the method generates and expands regulatory NK cells to numbers that suppress the immune-mediated disorder.

21. The method of claim 1, wherein the method generates and expands one or more lymphocyte populations to numbers that suppress the immune-mediated disorder.

22. The method of claim 1, wherein NK cells in the patient become TGF-β producing regulatory NK cells.

23. The method of claim 1, wherein T cells in the patient become TGF-β producing regulatory T cells.

24. The method of claim 15, wherein the cytokine is TGF-β and TGF-β is either encapsulated in the at least one synthetic polymeric nanoparticle or the at least one synthetic polymeric nanoparticle induces regulatory cells in vivo in the local environment.

25. The method of claim 1, wherein the immune-mediated disorder is at least one antibody-mediated autoimmune disease selected from the group consisting of: systemic lupus erythematosus, pemphigus vulgaris, myasthenia gravis, hemolytic anemia, thrombocytopenia purpura, Graves' disease, dermatomyositis, and Sjogren's disease.

26. The method of claim 1, wherein the immune-mediated disorder is at least one cell-mediated autoimmune disease selected from the group consisting of: type 1 Diabetes, Hashimoto's disease, polymyositis, inflammatory bowel disease, multiple sclerosis, rheumatoid arthritis, and scleroderma.

27. The method of claim 1, wherein the immune-mediated disorder is a graft-related disease.

28. The method of claim 1, wherein the immune-mediated disorder is rejection of a foreign organ transplant.

29. The method of claim 28, wherein the immune-mediated disorder is graft versus host disease.

30. The method of claim 1, wherein the method is performed in vitro.

31. The method of claim 1, wherein the method is performed in vivo.

32. The method of claim 1, wherein the administering to the patient is using parenteral delivery.

33. The method of claim 32, wherein the parenteral delivery is intravenous.

34. The method of claim 32, wherein the parenteral delivery is intramuscular.

35. The method of claim 32, wherein the parenteral delivery is subcutaneous.

36. The method of claim 1, wherein the administering to the patient is using oral delivery.

37. The method of claim 1, wherein the at least one synthetic polymeric nanoparticle is selected from the group consisting of: a glycide, a liposome, and a dendrimer.

38. The method of claim 1, wherein the aAPC is combined with at least one defensin.

39. The method of claim 38, wherein the at least one defensin comprises RTD-1.

40. The method of claim 16, wherein IL-2 is encapsulated.

41. The method of claim 15, wherein the at least one stimulating agent comprises IL-2 and TGF-β.

42. The method of claim 41, wherein at least one of IL-2 and TGF-β are encapsulated in the at least one synthetic polymeric nanoparticle.

43. A method of treating or preventing an immune-mediated disorder in a patient comprising administering to the patient: (i) a tolerogenic artificial Antigen Presenting Cell (aAPC) composition comprising: (a) at least one synthetic polymeric nanoparticle, (b) at least one targeting agent, and (c) at least one stimulating agent, wherein the at least one stimulating agent comprises at least one tolerogenic cytokine; and (ii) at least one anti-inflammatory agent.

44. The method of claim 43, wherein the at least one tolerogenic cytokine comprises IL-2, TGF-β, or a combination thereof.

45. The method of claim 44, wherein the at least one tolerogenic cytokine is encapsulated in the at least one synthetic polymeric nanoparticle.

46. The method of claim 43, wherein the at least one anti-inflammatory agent comprises at least one defensin.

47. The method of claim 46, wherein the at least one defensin comprises RTD-1.
